(19) United States
(12) Patent Application Publication
(10) Pub. No.: US 2026/0069690 A1
(43) Pub. Date: Mar. 12, 2026

(54) NOVEL CD4+ TUMOR INFILTRATING LYMPHOCYTES FOR THE TREATMENT OF CANCER

(71) Applicants: Shari PILON-THOMAS, Land O Lakes, FL (US); Amod SARNAIK, Tampa, FL (US); H. LEE MOFFITT CANCER CENTER AND RESEARCH INSTITUTE, INC., Tampa, FL (US); UNIVERSITY OF SOUTH FLORIDA, Tampa, FL (US)

(72) Inventors: Shari PILON-THOMAS, Land O Lakes, FL (US); Amod SARNAIK, Tampa, FL (US); MacLean HALL, St. Petersburg, FL (US); Daniel ABATE-DAGA, Tampa, FL (US)

(21) Appl. No.: 19/106,715

(22) PCT Filed: Aug. 28, 2023
(86) PCT No.: PCT/US2023/073017

§ 371 Date: Feb. 26, 2025

Related U.S. Application Data
(60) Provisional application No. 63/401,517, filed on Aug. 26, 2022.
(60) Provisional application No. 63/430,810, filed on Dec. 7, 2022.

(51) Int. Cl. A61K 40/11; A61K 40/32; A61K 40/42; A61P 35/00; C12N 5/0783

(52) U.S. Cl. CPC A61K 40/11; A61K 40/32; A61K 40/428; A61P 35/00; C12N 5/0636; C12N 2501/2302; C12N 2502/30

(57) ABSTRACT

Disclosed are novel adoptive cell therapies comprising engineered CD4+ T cells comprising novel T cell receptors and methods of their use in the treatment of cancer. Further, wherein an engineered CD4+ T cell comprising a T cell receptor (TCR) alpha (TCRa) chain comprising a complimentary determining region (CDR) 3 (CDR3) is disclosed.

### CROSS REFERENCE TO RELATED APPLICATIONS

This application claims the benefit of U.S. Provisional Application No. 63/401,517, filed on Aug. 26, 2022 and U.S. Provisional Application No. 63/430,810, filed on Dec. 7, 2022, application which are incorporated herein by reference in their entireties.

### STATEMENT OF GOVERNMENT SUPPORT

This invention was made with government support under Grant Nos. CA250320, CA178083, and CA076292 awarded by the National Institutes of Health. The government has certain rights in the invention.

### REFERENCE TO SEQUENCE LISTING

A Sequence Listing conforming to the rules of WIPO Standard ST.26 is hereby incorporated by reference. Said Sequence Listing has been filed as an electronic document via PatentCenter in ASCII format encoded as XML. The electronic document, created on Aug. 29, 2023, is entitled “10110-295 WO1.xml”, and is 28,672 bytes in size.

### I. BACKGROUND

Immunotherapy for cancer has long been focused on the generation of CD8+ cytotoxic T lymphocyte responses, independent of their dynamic CD4+ T cell counterpart. One promising approach, adoptive cell transfer (ACT) of tumor-infiltrating lymphocytes (TIL), has yielded response rates ranging from 28-55%. Although lasting and complete responses have been achieved, there is substantial opportunity for improvement. What are needed are new CD4+ T cell-based immunotherapies.

### II. SUMMARY

Disclosed are CD4+ T cells engineered to comprise a novel T cell receptor and methods of their use.

In one aspect, disclosed herein are engineered CD4+ T cells comprising a T cell receptor (TCR) alpha (TCRα) chain comprising a complimentary determining region (CDR) 3 (CDR3) as set forth in SEQ ID NO: 1, SEQ ID NO: 11, SEQ ID NO: 17, SEQ ID NO: 23, SEQ ID NO:29, SEQ ID NO: 35, or SEQ ID NO: 41 (such as, for example, a TCRα CDR3 encoded by the nucleic acid as set forth in SEQ ID NO: 2, SEQ ID NO: 12, SEQ ID NO: 18, SEQ ID NO: 24, SEQ ID NO: 30, SEQ ID NO: 36, or SEQ ID NO: 42, respectively) or the TCRα encoded by the nucleic acid as set forth in SEQ ID NO: 5, SEQ ID NO: 15, SEQ ID NO: 21, SEQ ID NO: 27, SEQ ID NO: 33, SEQ ID NO: 39, or SEQ ID NO: 45; and/or a T cell receptor (TCR) beta (TCRβ)chain comprising a complimentary determining region (CDR) 3 (CDR3) as set forth in SEQ ID NO: 3, SEQ ID NO: 13, SEQ ID NO: 19, SEQ ID NO: 25, SEQ ID NO: 31, SEQ ID NO: 37, or SEQ ID NO: 43 (such as, for example, a TCRβ CDR3 encoded by the nucleic acid as set forth in SEQ ID NO: 4, SEQ ID NO: 14, SEQ ID NO: 20, SEQ ID NO: 26, SEQ ID NO: 32, SEQ ID NO: 38, or SEQ ID NO: 44, respectively) or the TCR$ encoded by the nucleic acid as set forth in SEQ ID NO: 6, SEQ ID NO: 16, SEQ ID NO: 22, SEQ ID NO: 28, SEQ ID NO: 34, SEQ ID NO: 40, or SEQ ID NO: 46.

Also disclosed herein are methods of treating, inhibiting, reducing, decreasing, ameliorating, and/or preventing a cancer and/or metastasis in a subject comprising administering to the subject one or more of the engineered CD4+ T cells of any preceding aspect. For example, disclosed herein are methods treating, inhibiting, reducing, decreasing, ameliorating, and/or preventing a cancer and/or metastasis in a subject comprising administering to the subject an adoptive cell therapy comprising one or more engineered CD4+ T cells (including, but not limited to one or more engineered CD4+ T cell comprising a T cell receptor (TCR) alpha (TCRα) chain comprising a complimentary determining region (CDR) 3 (CDR3) as set forth in SEQ ID NO: 1, SEQ ID NO: 11, SEQ ID NO: 17, SEQ ID NO: 23, SEQ ID NO:29, SEQ ID NO: 35, or SEQ ID NO: 41 (such as, for example, a TCRα CDR3 encoded by the nucleic acid as set forth in SEQ ID NO: 2, SEQ ID NO: 12, SEQ ID NO: 18, SEQ ID NO: 24, SEQ ID NO: 30, SEQ ID NO: 36, or SEQ ID NO: 42, respectively) or the TCRα encoded by the nucleic acid as set forth in SEQ ID NO: 5. SEQ ID NO: 15, SEQ ID NO: 21, SEQ ID NO: 27, SEQ ID NO: 33, SEQ ID NO: 39, or SEQ ID NO: 45; and/or a T cell receptor (TCR) beta (TCRβ)chain comprising a complimentary determining region (CDR) 3 (CDR3) as set forth in SEQ ID NO: 3, SEQ ID NO: 13, SEQ ID NO: 19, SEQ ID NO: 25, SEQ ID NO: 31, SEQ ID NO: 37, or SEQ ID NO: 43 (such as, for example, a TCRβ CDR3 encoded by the nucleic acid as set forth in SEQ ID NO: 4, SEQ ID NO: 14, SEQ ID NO: 20, SEQ ID NO: 26, SEQ ID NO: 32, SEQ ID NO: 38, or SEQ ID NO: 44, respectively) or the TCRβ encoded by the nucleic acid as set forth in SEQ ID NO: 6, SEQ ID NO: 16, SEQ ID NO: 22, SEQ ID NO: 28, SEQ ID NO: 34, SEQ ID NO: 40, or SEQ ID NO: 46.

In one aspect, disclosed herein are disclosed herein are methods of treating, inhibiting, reducing, decreasing, ameliorating, and/or preventing a cancer and/or metastasis of any preceding aspect, wherein the CD4+ T cells are obtained from tumor infiltrating lymphocytes (TILs), marrow infiltrating lymphocytes (MILs), memory CD4+ T cells, Th1 CD4+ T cells, Th17 CD4+ T cells, Th2 CD4+ T cells, and regulatory CD4+ T cells (Tregs).

Also disclosed herein are disclosed herein are methods of treating, inhibiting, reducing, decreasing, ameliorating, and/or preventing a cancer and/or metastasis of any preceding aspect, wherein at least 80% of the cells transferred are CD4+ T cells.

In one aspect, disclosed herein are disclosed herein are methods of treating, inhibiting, reducing, decreasing, ameliorating, and/or preventing a cancer and/or metastasis of any preceding aspect, wherein the engineered CD4+ T cells are cultured in the presence of autologous tumor prior to administration to the subject.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows a schematic of adoptive cell therapy with tumor infiltrating lymphocytes (TILs).

FIGS. 2A and 2B show that CD4⁺ TILs persist in vivo. FIG. 2A shows CT scans of lung metastases from complete responder (CR) infused with predominantly CD4+ TIL. FIG. 2B shows persistence measured by TCRβ overlap between CD4+ and CD8+ TIL and weekly PBMC repertoire.

FIGS. 3A, 3B, and 3C show that CD8+ TILS fail to recognize tumors. FIG. 3A shows infusion product TILs were co-cultured with tumor followed by detection of released IFNg by ELISA. FIGS. 3B and 3C show that CD8+ TIL were isolated and co-cultured with HLA matched tumor +/−MHC Class I blocking antibody (W6/32). Tumor recognition was evaluated by IFNg release via ELISA and upregulation of cell surface activation markers by flow cytometry.

FIGS. 4A, 4B, 4C, and 4D show polyfunctional neoantigen-specific CD4+ TILs detected via immunogenic analysis. FIG. 4A shows mutant 25mers were predicted for MHC binding from whole exome sequencing. FIGS. 48, 4C, and 4D show that TIL were stimulated with peptides loaded on dendritic cells (DC) and assessed for increased cell surface expression of OX40 and 41BB by flow cytometry and effector molecule production by ELLA.

FIGS. 5A, 5B, 5C, and 5D show Clonal tracing demonstrates in vitro enrichment and in vivo persistence. FIG. 5A shows neoantigen-specific TIL were sorted on OX40/41BB expression (+/+ vs. −/−) in response to peptide. FIGS. 5B, 5C, and 5D show sorted and expanded (REP) TIL populations were sequenced at the TCRβ locus and tracked from infusion product across weekly patient peripheral blood samples for clonal persistence.

FIG. 6 shows that MSGV1 TCR Transgenic T cells specifically recognized mutant S100A11^{Q22R} peptide in the context of MHC Class II on B cells.

FIGS. 7A, 7B, 7C, 7D, 7E, 7F, 7G, 7H, and &I show CD4+ TIL exhibit effector response upon neoantigen peptide recognition. FIG. 7A shows treatment course of ACT for Patient 1, accompanied by CT images of target lesions (red circles) before and after therapy. D=day (relative to TIL infusion), Sx=surgery, CT=computerized tomography scan, TL=target lesion. FIG. 7B shows measurement of the longest diameter of each target lesion (upper) and sum of target lesions (lower) during patient follow-up window. Red dotted line indicates a −30% change from the baseline sum. FIG. 7C shows all neoantigen peptides (n=65) were prioritized by additive score (AS). Blue arrow indicates peptide 60 (P60). See Methods for detailed explanation. ES=expression score, MHC-II=mhc2_score, MHC-I=mhc1_score, AS=additive score. FIG. 7D shows bulk TIL were co-cultured with DC loaded with the neoantigen peptide pool (PP) or no peptide (NP), then stained for cell sorting by OX40 and 4-1BB induction. FIG. 7E shows sort co-culture supernatants were assayed for TIL production of granzyme B (GZMB), IFNg, and TNFa via the Ella system. FIG. 7F shows TIL were validated for individual neoantigen peptide recognition by flow cytometric evaluation of OX40 and 4-1BB expression (upper) and IFNg release (lower) in co-culture supernatants. FIG. 7G shows nriched CD4+ TIL were assayed for effector molecule secretion in response to stimulation with P60 or no stimulation (TIL). FIG. 7H shows utant (P60^{MUT}=P60) and wildtype (P60^{WT}) versions of P60 were loaded onto APCs and utilized to stimulate enriched CD4+ TIL. IFNg release was quantified in co-culture supernatants. FIG. 7I shows that following co-culture of enriched TIL with P60 or NP, CD4+ TIL were stained for flow cytometric analysis of the indicated cell surface and intracellular molecules.

FIG. 8A shows bulk infusion product (left) or CD8-enriched (right) TIL from Patient 1 were cultured alone (TIL Only), with autologous tumor (AT), with HLA-matched tumor (HLA MT), or with anti-CD3 antibody (clone OKT3). IFNg production was measured by ELISA. Mean+SD.

FIG. 8B shows CD8-enriched from Patient 1 were co-cultured overnight as in (A), then stained with antibodies for PD-1 and 4-1BB and analyzed by flow cytometry. The MHC Class I blocking antibody (W6/32) was utilized where indicated.

FIG. 9A shows DNA and RNA were extracted from tumor blocks from Patient 1 and sequenced at the S100A11 locus. Nucleic acid conversion is represented by the color change from red (T) to blue (C) resulting in the Q22R nonsynonymous mutation.

FIG. 9B shows MHC Class II expression in tumor from Patient 1 by RNA sequencing. Fragments per kilobase of exon per million mapped fragments (FPKM).

FIGS. 10A, 10B, 10C, and 10D show CD4⁺ TIL demonstrate cytotoxic potential restricted by TCR-HLA-DR interaction. FIG. 10A shows enriched CD4⁺ TIL were co-cultured in the indicated conditions and cell culture supernatants were analyzed for granzyme B, IFNg, and TNFa production. FIG. 10B shows live cell imaging of TIL (unlabeled) and target cells (green) loaded with WT or mutated (Q22R) S100A11 peptide co-cultured at indicated effector:target (E:T) ratios for six hours. Still image acquired at four-hour time point with 10:1 E:T. Cleaved caspase 3/7 (red) induction was monitored via imaging at regular 30-minute intervals and quantified for target cell count (bottom right) and overlap of target cells and cleaved caspase 3/7 (bottom left). Unl=Unloaded targets. FIGS. 10C and 10D show TCR-T or UT peripheral blood lymphocytes (PBL) were co-cultured with B cells loaded with S100A11 peptides and blocking antibodies. Effector molecule production was quantified in cell co-culture supernatants (10C) and cell surface OX40 and 4-1BB expression was evaluated by flow cytometry (10D).

FIG. 11A shows overlapping custom peptides 12 to 16 amino acids in length spanning the S100A11^{Q22R} 25mer were synthesized and utilized to stimulate TIL from Patient 1. IFNg secretion and OX40 fold change in median fluorescence intensity (MFI) over control were quantified and mapped to the corresponding peptides.

FIG. 11B shows time lapse of the live cell imaging assay reported in FIG. 10B. TIL (unlabeled) and target cells (green) were loaded with WT or mutated S100A11^{Q22R} peptide and co-cultured for six hours at a 10:1 E:T ratio.

FIG. 11C shows clonal tracking of the top T cell clones in the infusion product and sorted TIL fractions by TCRb sequencing. The S100A11^{Q22R}-specific TIL clone is shaded blue.

FIGS. 12A, 12B, 12C, 12D, 12E, 12F, and 12G show clonal analysis of neoantigen-specific CD4⁺ TIL indicates persistence and effector profile. FIG. 12A showsTCRb sequencing of the infused TIL product for Patient 1 displayed as relative productive frequency. Blue slice is the S100A11^{Q22R}-specific clone. FIG. 12B shows relative productive frequency of the S100A11^{Q22R}-specific clone tracked longitudinally in PBMC samples at the indicated weeks (W) following TIL infusion. FIG. 12C shows uniform manifold approximation and projection (UMAP) of the S100A11^{Q22R}-specific CD4⁺ TIL clone (left) and the residual CD4⁺ TIL (rCD4) from Patient 1. Cells were colored by clusters. FIG. 12D shows relative frequency of the clusters displayed in FIG. 12C. FIG. 12E shows scaled average expression of reported genes associated with neoantigen specificity in S100A11^{Q22R}-specific CD4⁺ TIL and rCD4 TIL. Hierarchical clustering was performed on the selected genes and cell types. FIG. 12F shows differentially expressed genes (DEG) between the S100A11^{Q22R}-specific CD4+ TIL and rCD4 cells, separated by clusters. FIG. 12G shows gene set enrichment analysis (GSEA) using DEG comparing S100A11^{Q22R}-specific CD4+ TIL clone versus rCD4 from Patient 1 across clusters. Normalized enrichment scores (NES) for pathway analysis were displayed. Hallmark, canonical pathways (CP) and gene ontogeny (GO) data sets were utilized.

FIGS. 13A, 13B, and 13C show a UMAP from scRNASeq analysis of cell clusters (13A) and the distribution of patient samples (13B) and TCR expression (13C).

FIGS. 13D, 13E, and 13F. Cell cluster annotation by canonical genes reported in the literature (13D), differential gene expression (13E), and reported genes of neoantigen-specific CD4⁺ T cells (13F).

FIGS. 14A, 14B, 14C, 14D, 14E, 14F, 14G, 14H, 14I, and 14J show Multiple CD4+ TIL clones display distinct neoantigen reactivity. FIG. 14A shows treatment course and CT images of target lesions (red circles) before and after therapy for Patient 2. D=day (relative to TIL infusion), Sx=surgery, CT=computerized tomography scan, TL=target lesion. FIG. 14B shows individual (upper) and sum (lower) measurements of target lesions by longest diameter for Patient 2. Red dotted line indicates a −30% change from the baseline sum. FIG. 14C shows bulk TIL and DC loaded with the neoantigen peptide pool (PP) or no peptide (NP) were co-cultured followed by surface staining of OX40 and 4-1BB for cell sorting. FIG. 14D shows bulk TIL production of granzyme B (GZMB), IFNg, and TNFa was analyzed in sort co-culture supematants. FIG. 14E shows analysis of IFNg release by TIL in response to individual neoantigen peptides loaded onto APCs. Gray area were below threshold of reactivity. FIG. 14F shows TIL (unlabeled) and neoantigen-loaded target cells (green) were co-cultured for six hours in a live cell imaging assay to capture cleaved caspase 3/7 (red) induction. Representative image displayed at four hours of co-culture with 10:1 E:T. FIG. 14G shows real-time cell-analysis (RTCA) of IFNg-pretreated autologous tumor (ATγ) following addition of neoantigen-specific CD4⁺ TIL. FIG. 14H shows APCs were loaded with neoantigen peptides, pre-coated with HLA-DR or HLA-DP blocking antibodies, and co-cultured with neoantigen-specific CD4+ TIL. IFNg release was quantified by the Ella system for each condition. FIG. 14I shows clonal tracking by TCRb sequencing of neoantigen-specific CD4+ TIL clones in the infused TIL product and PBMC at the indicated weeks (W). FIG. 14J show the distribution of scRNASeq cell clusters present in neoantigen-specific CD4+ TIL clones, identified by TCRb sequence.

FIG. 15A shows infusion product bulk TIL from Patient 2 were co-cultured with AT, HLA-Matched, and HLA-Mismatched tumor. Cell culture supernatants were assessed for IFNg release by ELISA.

FIG. 15B shows TILs were enriched by IFNg capture and stimulated with AT or IFNg pre-conditioned AT (ATg). Cytokine production was determined by intracellular flow cytometry staining in CD4+ TIL.

FIG. 15C shows that neoantigen peptides were prioritized by additive score (AS). Blue arrows and annotation indicate peptides with observed T cell reactivity. ES=expression score, MHC-II=mhc2_score, MHC-I=mhc1_score, AS=additive score.

FIG. 15D shows Flow cytometric analysis of live singlets gated on CD3+ T cells in the bulk infusion product sample and following cell sorting of TIL that upregulated 4-1BB and OX40 in response to pooled neoantigen peptides.

FIG. 15E shows time lapse of the live cell imaging assay reported in FIG. 14F. TIL (unlabeled) and target cells (green) were loaded with WT or mutated TNS1P694S peptide and co-cultured for six hours at a 10:1 E:T ratio.

FIG. 15F shows scaled average expression of reported genes associated with neoantigen specificity in NeoAg CD4+ TIL and nCD4 TIL. Hierarchical clustering was performed on the selected genes and cell types.

FIG. 16A shows infusion product bulk TIL from Patient 3 were co-cultured with AT and HLA-Matched tumor. Cell culture supernatants were assessed for IFNg release by ELISA.

FIG. 16B shows neoantigen peptides from Patients 3 (PT3) and 4 (PT4) were prioritized by additive score (AS). Blue arrows and annotation indicate peptides with observed T cell reactivity. ES=expression score, MHC-II=mhc2_score, MHC-I=mhc1_score, AS=additive score.

FIG. 16C shows CD4 and CD8 expression of neoantigen-enriched TIL from Patient 3 following cell sorting on 4-1BB and OX40 expression and again after magnetic enrichment of CD4+ T cells.

FIG. 16D shows AT from Patient 3 was transduced with the CIITA expression vector and loaded with the indicated neoantigen peptides. Effector molecule secretion from CD4+ TIL was quantified via the Ella platform.

FIG. 17A, 17B, 17C, 17D, 17E, 17F, 17G, 17H, 17I, 17J, and 17K show TIL product from non-responders contains neoantigen-specific CD4+ T cells. FIGS. 17A and 17F show bulk TIL from Patient 3 (17A) and Patient 4 (17F) were co-cultured with autologous DC loaded with neoantigen peptide pool (PP) or no peptide (NP). OX40 and 4-1BB induction was analyzed by flow cytometry on the indicated T cell populations. FIGS. 17B and 17G show granzyme B (GZMB), IFNg, and TNFa were quantified in co-culture supernatants from Patient 3 (17B) and Patient 4 (17G). FIGS. 17C and 17H show sorted TIL were validated for individual neoantigen peptide recognition by IFNg release following co-culture with peptide-loaded autologous B cells, respectively from Patient 3 (17C) and Patient 4 (17H). FIGS. 17d and 17I show neoantigen peptide hits were loaded on autologous B cells, pretreated with the indicated blocking antibodies, then co-cultured with neoantigen-specific CD4+ TIL to assess IFNg production from Patient 3 (17D) and Patient 4 (17I). FIG. 17E shows effector molecule secretion and OX40 and 4-1BB induction on neoantigen-specific CD4+ TIL from Patient 3 were quantified in response to AT-CIITA loaded with neoantigen peptide hits. FIG. 17J shows clonal frequency of neoantigen-specific CD4+ TIL from Patient 4 in the infused TIL product and PBMC at the indicated weeks (W) following ACT. FIG. 17K shows the frequency of the scRNASeq cell clusters present within neoantigen-specific CD4+ TIL clones from Patient 4.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10”as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular CD4+ TCR is disclosed and discussed and a number of modifications that can be made to a number of molecules including the CD4+ TCR are discussed, specifically contemplated is each and every combination and permutation of CD4+ TCR and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

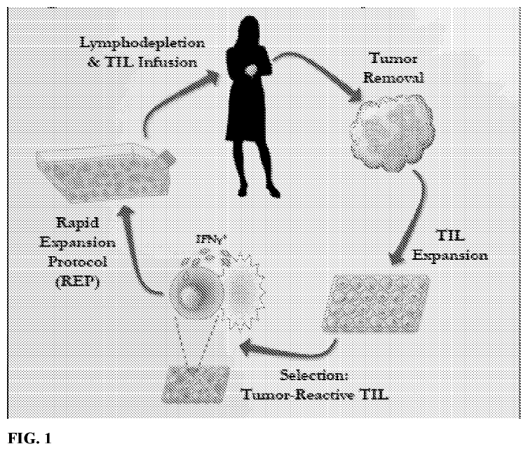

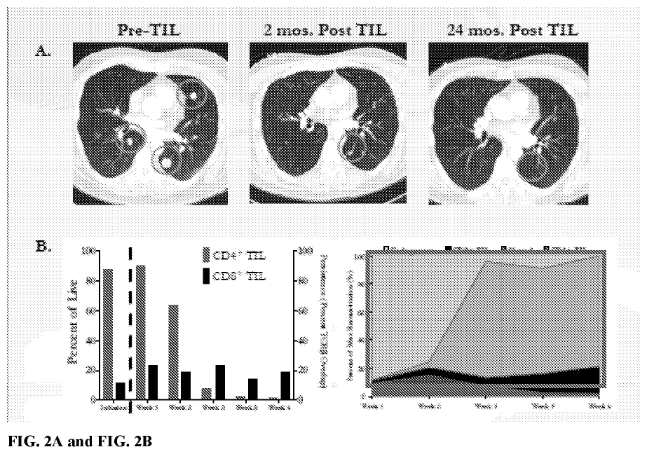

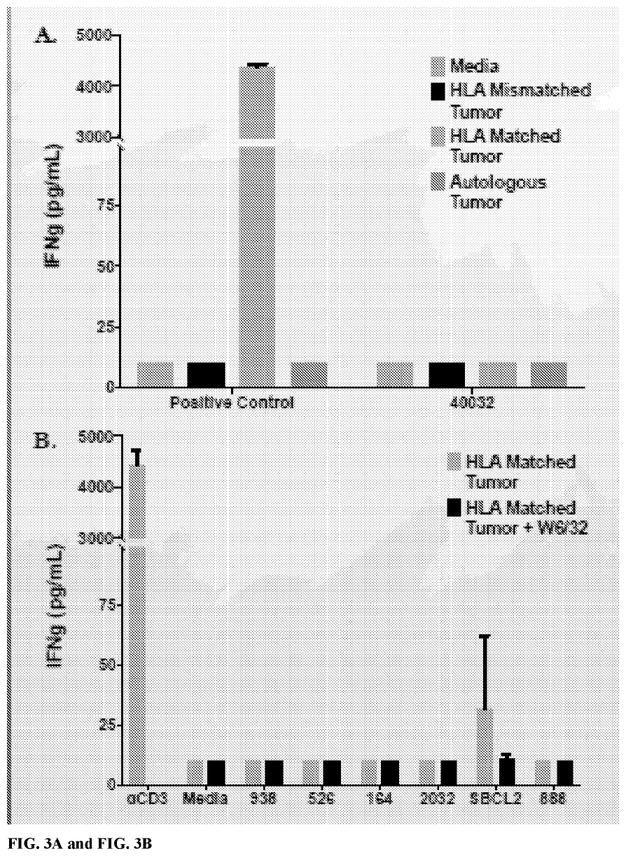

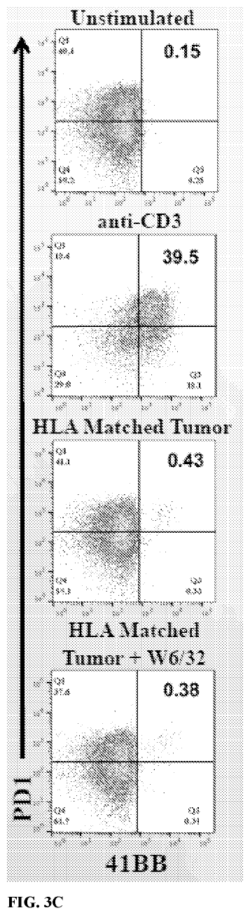

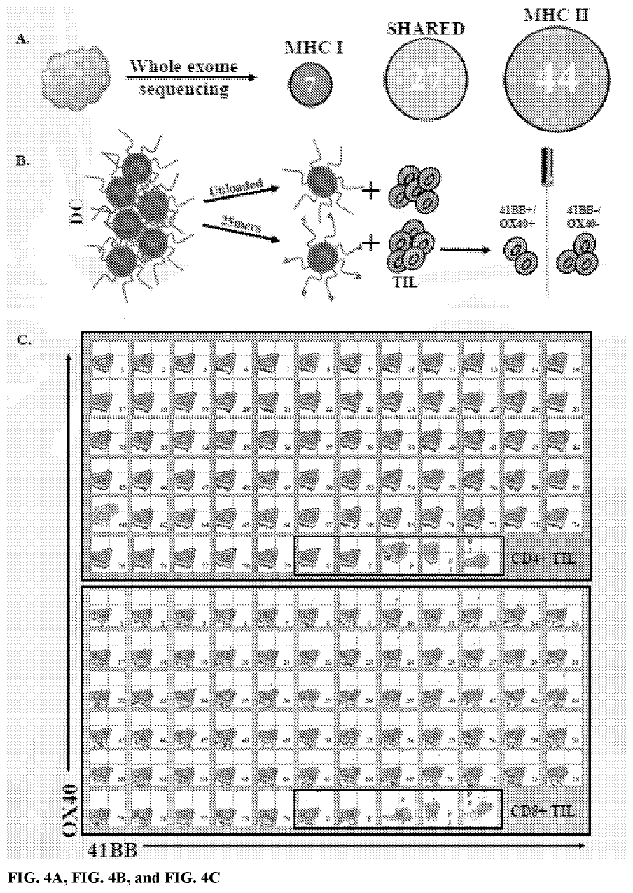

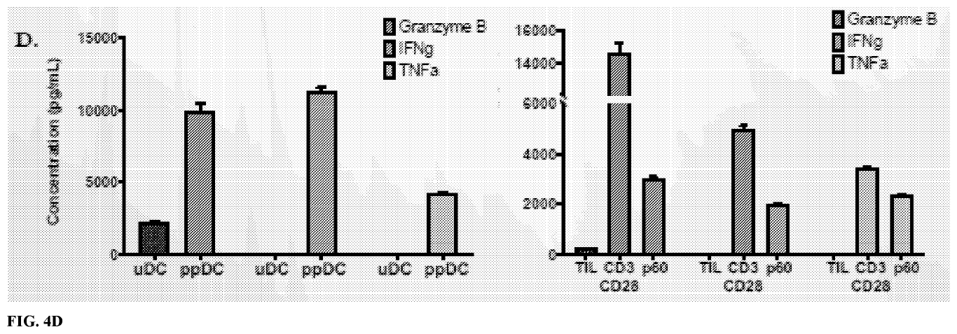

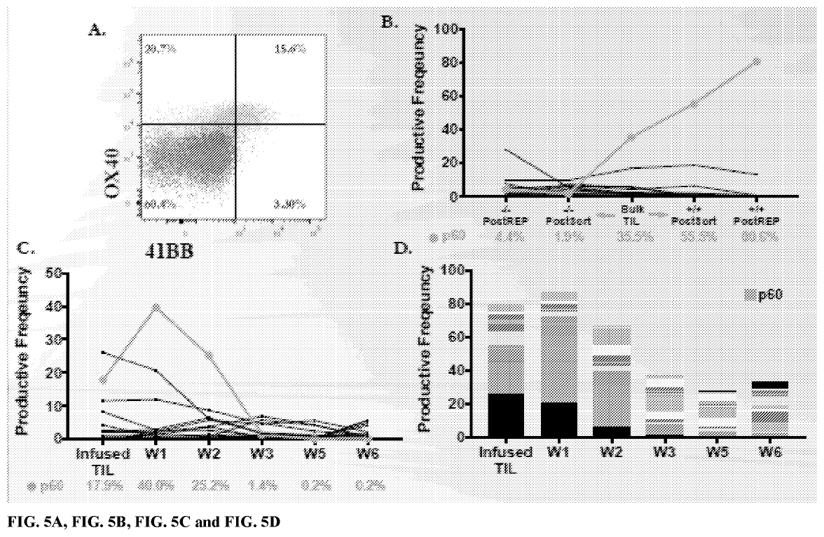

Investigation into the role of CD4+ TIL in the adoptive cell transfer (ACT) setting (FIG. 1) remains critically underexplored. CD4+ T cells recognize tumor antigen presented on MHC Class II either directly on tumor cells or indirectly through antigen presenting cells (APCs) and are able to elicit potent anti-tumor responses under the appropriate conditions. Here, we present a case study of a metastatic melanoma patient who received adoptive transfer of a predominantly (88%) CD4+ TIL product. This patient demonstrated a complete response (CR) to therapy despite a lack of detection of IFNg in the infusion product in vitro when these TIL were cocultured with autologous tumor prior to ACT (FIG. 3A). Tumor recognition was also absent when CD8+ TIL were isolated and stimulated directly with HLA matched tumor lines, indicating a lack of recognition of shared melanoma antigens presented on MHC Class I (FIG. 3B). Longitudinal analysis of the peripheral blood of this patient confirmed that the infused CD4+ TIL persisted after therapy for at least six weeks (FIG. 2). Whole exome sequencing (WES) performed on the TIL surgical specimen discovered 88 non-synonymous single nucleotide variants (SNVs) as candidate neoantigens (FIG. 4A). Predicted binding of the resulting mutant peptides to autologous HLA molecules generated a predominantly MHC Class II restricted profile, with 81.8% of variants capable of MHC Class II presentation and greater than half exclusive to MHC Class II only. CD4+ TIL were screened for tumor antigen recognition by upregulation of OX40 and 41BB after stimulation with autologous APCs loaded with mutant peptides (FIGS. 4B, 4C, and 4D). Nearly half (49.2%) of CD4+ TIL responded to tumor-derived peptides. These CD4+ TIL were then sorted into tumor-reactive and non-reactive subsets for further clonal analysis of phenotype and transcriptional profile (scRNASeq) of these T cells in order to characterize the nature of the CD4+ TIL response to tumor antigen (FIG. 5). Overall, thorough interrogation of this patient's case study demonstrated evidence of CD4+ TIL involvement in a complete clinical response after ACT.

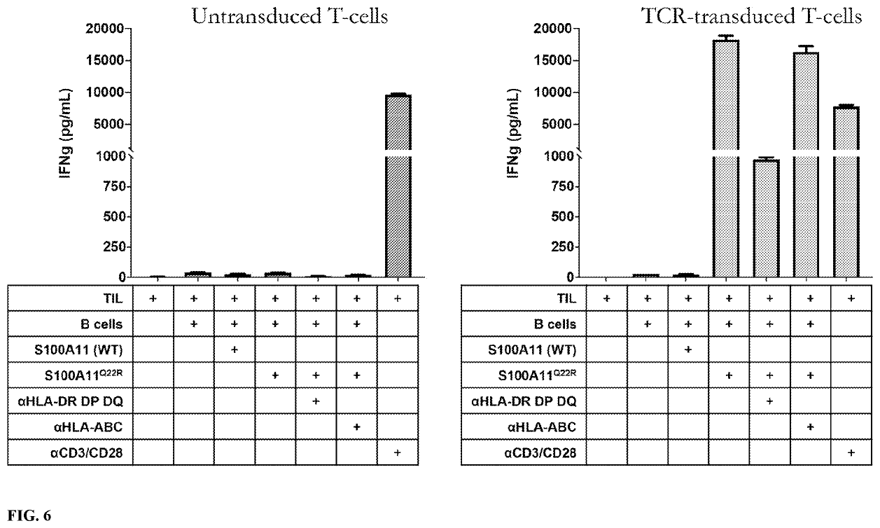

As shown in FIG. 6, TCR-transduced peripheral blood T lymphocytes recognize the S100A11^{Q22R} mutation in the context of class-II presentation. Peripheral blood T cells were expanded from a healthy donor, and transduced with a retroviral vector (MSGV1) encoding an S100A11^{Q22R}-specific TCR. Untransduced expanded T cells were used as negative controls. Upon coculture with autologous B cells pulsed with an S100A11-derived peptide that contain the Q22R mutation, TCR-transduced T-cells secreted more than 15,000 pg/mL of IFNg. This secretion was inhibited by addition of an anti-HLA-DR/-DP/-DQ antibody, but not by an anti-HLA-ABC antibody, confirming that this TCR is HLA Class-II-restricted. Polyclonal stimulation with anti-CD3/-CD28 antibodies was used as a positive control of T cell activation.

In one aspect, disclosed herein are engineered CD4+ T cells (including, but not limited to the CD4+ T cells obtained from tumor infiltrating lymphocytes (TILs), marrow infiltrating lymphocytes (MILs), memory CD4+ T cells, Th1 CD4+ T cells, Th17 CD4+ T cells, Th2 CD4+ T cells, and/or CD4+ T cells (Tregs)) comprising a T cell receptor (TCR) alpha (TCRα) chain comprising a complimentary determining region (CDR) 3 (CDR3) as set forth in SEQ ID NO: 1, SEQ ID NO: 11, SEQ ID NO: 17, SEQ ID NO: 23, SEQ ID NO:29, SEQ ID NO: 35, or SEQ ID NO: 41 (such as, for example, a TCRα CDR3 encoded by the nucleic acid as set forth in SEQ ID NO: 2. SEQ ID NO: 12, SEQ ID NO: 18, SEQ ID NO: 24, SEQ ID NO: 30, SEQ ID NO: 36, or SEQ ID NO: 42, respectively) or the TCRα encoded by the nucleic acid as set forth in SEQ ID NO: 5, SEQ ID NO: 15, SEQ ID NO: 21, SEQ ID NO: 27, SEQ ID NO: 33, SEQ ID NO: 39, or SEQ ID NO: 45; and/or a T cell receptor (TCR) beta (TCRβ)chain comprising a complimentary determining region (CDR) 3 (CDR3) as set forth in SEQ ID NO: 3, SEQ ID NO: 13, SEQ ID NO: 19, SEQ ID NO: 25, SEQ ID NO: 31, SEQ ID NO: 37, or SEQ ID NO: 43 (such as, for example, a TCRβ CDR3 encoded by the nucleic acid as set forth in SEQ ID NO: 4, SEQ ID NO: 14, SEQ ID NO: 20, SEQ ID NO: 26, SEQ ID NO: 32, SEQ ID NO: 38, or SEQ ID NO: 44, respectively) or the TCRβ encoded by the nucleic acid as set forth in SEQ ID NO: 6, SEQ ID NO: 16, SEQ ID NO: 22, SEQ ID NO: 28, SEQ ID NO: 34, SEQ ID NO: 40, or SEQ ID NO: 46.

### 1. Homology/Identity

It is understood that one way to define any known variants and derivatives or those that might arise, of the disclosed genes and proteins herein is through defining the variants and derivatives in terms of homology to specific known sequences. For example, SEQ ID NO: 2 sets forth a particular sequence of a complimentary determining region 3 (CDR3) of a T cell receptor (TCR) alpha (TCRα) and SEQ ID NO: 2 sets forth a particular sequence of the peptide encoded by SEQ ID NO: 1. Specifically disclosed are variants of these and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins or nucleic acids, such as genes. For example, the homology can be calculated after aligning the two sequences so that the homology is at its highest level. Another way of calculating homology can be performed by published algorithms.

Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzyinol. 183:281-306, 1989 which are herein incorporated by reference for at least material related to nucleic acid alignment.

### 2. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, PA Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### C. Methods of Using the Compositions

### 1. Method of Treating Cancer

It is understood and herein contemplated that the engineered CD4+ T cells disclosd herein can be used in the treatment of cancer. According, disclosed herein are methods of treating, inhibiting, reducing, decreasing, ameliorating, and/or preventing a cancer and/or metastasis in a subject comprising administering to the subject one or more of the engineered CD4+ T cells disclosed herein. For example, disclosed herein are methods treating, inhibiting, reducing, decreasing, ameliorating, and/or preventing a cancer and/or metastasis in a subject comprising administering to the subject an adoptive cell therapy comprising one or more engineered CD4+ T cells (including, but not limited to one or more engineered CD4+ T cell comprising a T cell receptor (TCR) alpha (TCRα) chain comprising a complimentary determining region (CDR) 3 (CDR3) as set forth in SEQ ID NO: 1, SEQ ID NO: 11, SEQ ID NO: 17, SEQ ID NO: 23, SEQ ID NO:29, SEQ ID NO: 35, or SEQ ID NO: 41 (such as, for example, a TCRα CDR3 encoded by the nucleic acid as set forth in SEQ ID NO: 2, SEQ ID NO: 12, SEQ ID NO: 18, SEQ ID NO: 24, SEQ ID NO: 30, SEQ ID NO: 36, or SEQ ID NO: 42, respectively) or the TCRα encoded by the nucleic acid as set forth in SEQ ID NO: 5, SEQ ID NO: 15, SEQ ID NO: 21, SEQ ID NO: 27, SEQ ID NO: 33, SEQ ID NO: 39, or SEQ ID NO: 45; and/or a T cell receptor (TCR) beta (TCRβ)chain comprising a complimentary determining region (CDR) 3 (CDR3) as set forth in SEQ ID NO: 3, SEQ ID NO: 13, SEQ ID NO: 19, SEQ ID NO: 25, SEQ ID NO: 31, SEQ ID NO: 37, or SEQ ID NO: 43 (such as, for example, a TCRβ CDR3 encoded by the nucleic acid as set forth in SEQ ID NO: 4, SEQ ID NO: 14, SEQ ID NO: 20, SEQ ID NO: 26, SEQ ID NO: 32, SEQ ID NO: 38, or SEQ ID NO: 44, respectively) or the TCRβ encoded by the nucleic acid as set forth in SEQ ID NO: 6, SEQ ID NO: 16, SEQ ID NO: 22, SEQ ID NO: 28, SEQ ID NO: 34, SEQ ID NO: 40, or SEQ ID NO: 46. In one aspect, the CD4+ Cells are obtained from tumor infiltrating lymphocytes (TILs), marrow infiltrating lymphocytes (MILs), memory CD4+ T cells, Th1 CD4+ T cells, Th17 CD4+ T cells. Th2 CD4+ T cells, and regulatory CD4+ T cells (Tregs).

Also disclosed herein are disclosed herein are methods of treating, inhibiting, reducing, decreasing, ameliorating, and/or preventing a cancer and/or metastasis, wherein at least 80% of the cells transferred are CD4+ T cells.

It is understood and herein contemplated that the engineered CD4+ T cells can be cultured with autologous tumor from the recipient subject (i.e., the subject being treated) to prime the CD4 T cells prior to administration. Thus, in one aspect, disclosed herein are disclosed herein are methods of treating, inhibiting, reducing, decreasing, ameliorating, and/or preventing a cancer and/or metastasis, wherein the engineered CD4+ T cells are cultured in the presence of autologous tumor prior to administration to the subject. Culture of the CD4+ T cells in the presence of autologous tumor can occur for any time sufficient to prime the engineered CD4+ T cells, including, but not limited to 5, 10, 15, 20, 25, 30,, 35, 40, 45, 50, 55, 60, 65, 70, 75, 80, 85, 90, 95, 100, 105, 110, 115, 120, 130, 140, 150, 160, 170, 180 min, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 hours, 3, 4, 5, 6, 7, 8, 9, 10, 11, 12, 13, or 14 days.

The disclosed compositions can be used to treat any disease where uncontrolled cellular proliferation occurs such as cancers. A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer.

In one aspect, it is understood and herein contemplated that successful treatment of a cancer in a subject is important and doing so may include the administration of additional treatments. Thus, the disclosed methods of treating, reducing, inhibiting, decreasing, ameliorating and/or preventing a cancer and/or metastasis can include or further include any anti-cancer therapy known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane),Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil-Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel. Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epimbicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil-Topical), Fluorouracil Injection, Fluorouracil-Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Tfosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin. Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, ,Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil-Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). The treatment methods can include or further include checkpoint inhibitors include, but are not limited to, antibodies that block PD-1 (Nivolumab (BMS-936558 or MDX1106), CT-011, MK-3475), PD-L1 (MDX-1105 (BMS-936559), MPDL3280A, or MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016).

### D. Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Novel CD4+ Tumor Infiltrating Lymphocytes for the Treatment of Cancer

Immunotherapy has revolutionized the expected outcomes for cancer patients due to the potential for durable, complete clinical responses, yet there remains a dire need to expand and improve upon these therapies. In metastatic melanoma, adoptive cell transfer (ACT) with tumor-infiltrating lymphocytes (TIL) has resulted in objective response rates ranging from 28-56%. Most current TIL production strategies focus on the expansion and selection of tumor-reactive CD8+ T cells for infusion. However, recent evidence indicates an expanded role for CD4+ T cells in anti-tumor immunity, warranting further investigation into the function and efficacy of this underexplored TIL population in ACT.

Shared antigen and neoantigen-specific CD4⁺ T cells have been detected in the peripheral blood and within the tumor microenvironment across multiple cancer types, indicating their availability and utility in the immunotherapeutic setting. Several notable studies have demonstrated the efficacy of antigen-specific CD4⁺ T cells in ACT, including complete responses (CR) in advanced cancers following infusion of highly potent CD4-dominant T cell products. These clinical responses were marked by CD4⁺ effector T cells with pleiotropic functionality including cytokine production and long-term persistence, resulting in overall reduced tumor burden. While these reports indicated that antigen-specific CD4′ T cells were effective mediators of the anti-tumor immune response, the prevalence, profile, and mechanism of action remain unclear.

CD4⁺ T cells are integral orchestrators of both the primary and secondary immune responses, yet have been classically considered helpers. A diverse and plastic population, CD4⁺ T cells are capable of a polyfunctional response upon recognition of cognate peptide presented on MHC Class II molecules on antigen presenting cells (APCs) or tumor cells directly. Recent reports have demonstrated that CD4⁺ T cells possess direct and indirect effector functions, including pleiotropic cytokine production and tumor cytolytic capacity dependent on MHC Class II-directed cell-to-cell interactions resulting in T cell degranulation. The maintenance of these antigen-specific CD4⁺ T cells in vivo is critical to therapeutic efficacy and has been demonstrated in long-term responders following ACT. The advent of single cell RNA sequencing (scRNASeq) has improved the depth and insight into neoantigen-specific T cells dramatically, uncovering genetic signatures and modules which demarcate this critical population.

Here, we report the identification and characterization of neoantigen-specific CD4+ T cell clones present within the TIL infusion product from previously treated melanoma patients. Through a series of case studies, we demonstrated that these neoantigen-specific CD4+ TIL were highly activated in response to tumor antigens and exhibited MHC Class II-mediated cytotoxicity. We then utilized T cell receptor (TCR) sequencing and scRNASeq to validate the in vitro and in vivo functional activity and further contextualize these neoantigen-specific CD4+ TIL. Overall, these data support an important role for neoantigen-specific CD4+ TIL and advocate for their inclusion in ACT.

### a) Materials and Methods

### (1) Patients and Patient Samples

Retrospective analysis was performed on TIL, peripheral blood mononuclear cells (PBMC), and excess tumor material derived from metastatic melanoma patients previously treated with ACT at Moffitt Cancer Center (MCC) under the clinical trial protocols NCT01005745 (Patients 1, 2, 5), NCT01701674 (Patient 3), and NCT01659151 (Patients 4 and 6). As necessary, TIL were propagated by the Rapid Expansion Protocol (REP), consistent with prior publications. CD4+ TIL were enriched by negative selection utilizing CD8 microbeads (Miltenyi Biotec, Gaithersburg, MD) or positive selection utilizing the IFNg Secretion Assay—Detection Kit (PE) (Miltenyi Biotec) in combination with anti-PE MicroBeads (Miltenyi Biotec) following the manufacturer's protocol where indicated.

### (2) DNA/RNA Extraction for Neoantigen Detection

Additional excess tumor tissue was preserved as formalin-fixed, paraffin-embedded (FFPE) or snap frozen (SF) tumor blocks within 15 minutes of surgical extirpation. FFPE tissues were placed in 10% neutral buffered formalin and fixed for 24 hours prior to embedding in paraffin using standard methodologies. SF tissues were immediately frozen and stored in liquid nitrogen until processing. Sections (4 uM) from each tumor block were stained with hematoxylin and eosin (H&E) and reviewed by the study pathologist to ensure tumor content. DNA and RNA from Patient 1 were extracted with the QIAamp DNA FFPE Tissue Kit (Qiagen Sciences, Inc., Germantown, MD) and Ambion RecoverAll Kit (ThermoFisher Scientific), respectively. RNA for this patient was DNase treated, followed by cleanup with the RNeasy MinElute Cleanup Kit (Qiagen Sciences, Inc). For all other patients, DNA and RNA were extracted with the Allprep DNA/RNA kit (Qiagen Sciences, Inc.). Genetic material was quantified by Qubit fluorometric quantification (ThermoFisher Scientific, Waltham, MA) and quality control was performed utilizing the TapeStation 4200 System (Agilent Technologies, Inc., Santa Clara, CA).

### (3) Whole-Exome Sequencing/RNA-Sequencing

### (a) Patient 1

Whole-exome sequencing (WES) was performed on DNA from fixed tumor tissue (FFPE) and from blood as a germline control in order to identify somatic mutations in the coding regions of the human genome. Following a quantitative-PCR (qPCR)-based DNA quality and quantity assessment using the Agilent NGS FFPE QC Kit with the tumor DNA sample, 200 ng of DNA was used as input into the Agilent SureSelect XT Clinical Research Exome kit (Agilent Technologies). Briefly, for each tumor DNA sample, a genomic DNA library was constructed according to the manufacturer's protocol and the size and quality of the library was evaluated using the Agilent BioAnalyzer. An equimolar amount of library DNA was used for a whole-exome enrichment using the Agilent capture baits and after qPCR library quantitation and quality control (QC) analysis on the BioAnalyzer, approximately 200 million and 80 million 75-base paired-end sequences for the tumor and germline control sample, respectively, were generated using v2 chemistry on an Illumina NextSeq 500 high-output sequencing run (Illumina, Inc., San Diego, CA).

An RNA-sequencing (RNASeq) library was prepared using the Illumina TruSeq RNA Exome Library Preparation Kit (Illumina, Inc.) according to the manufacturer's protocol. Briefly, following RNA quality review on the Agilent TapeStation (Agilent Technologies) and quantitation with the Qubit RNA BR Assay Kit (ThermoFisher Scientific), 100 ng of RNA was used as input RNA fragmentation. The cDNA libraries were generated according to the protocol and reviewed for quality and quantity using the Qubit dsDNA Assay Kit and the Agilent BioAnalyzer DNA 1000 Chip (Agilent Technologies). The library was then enriched twice using the Illumina exome probes. The final library was reviewed for initial quantity and quality using the Qubit dsDNA Assay Kit (ThermoFisher Scientific) and the BioAnalyzer High Sensitivity DNA Chip (Agilent Technologies). Following library quantitation with the Kapa Library Quantification Kit for NGS (Roche Sequencing, Pleasanton, CA), the library was sequenced on an Illumina NextSeq 500 (Illumina, Inc.) mid-output run to generate 174M million pairs of 75-base reads.

For confirmation of the expression of the S100A11^{Q22R} mutation in the FFPE tumor tissue from Patient 1, RNA was extracted and DNase treated prior to processing with the Nugen RNASeq Universal Kit (Tecan US, Inc., Morrisville, NC). The generated cDNA was quantified and reviewed for quality control metrics, then sequenced on the NextSeq 500 (Illumina, Inc.) 2×75 base pair mid-output run and analyzed for nucleic acid conversion (T>C) at position Chr1 152006215 in the human genome version hs37d5.

### (b) Patient 2

WES was performed on DNA from frozen tumor tissue and from blood as a germline control in order to identify somatic mutations in the coding regions of the human genome. Briefly, 200 ng of DNA was used as input into the Agilent SureSelect XT Clinical Research Exome kit (Agilent Technologies). For each tumor DNA sample, a genomic DNA library was constructed according to the manufacturer's protocol and the size and quality of the library was evaluated using the Agilent BioAnalyzer. An equimolar amount of library DNA was used for a whole-exome enrichment using the Agilent capture baits and after qPCR library quantitation and QC analysis on the BioAnalyzer. Approximately 195 million and 134 million 75-base paired-end sequences for the tumor and germline control sample, respectively, were generated using v2 chemistry on an Illumina NextSeq 500 high-output sequencing run (Illumina, Inc.).

An RNAseq library was prepared using the NuGen FFPE RNA-Seq Multiplex System (later renamed to Universal RNA-Seq Library Preparation Kit with NuQuant, (Tecan US, Inc.). DNase-treated RNA (100 ng) was used to generate cDNA and a strand-specific library following the manufacturer's protocol. Library molecules containing ribosomal RNA sequences were depleted using the NuGen AnyDeplete probe-based enzymatic process. The final library was assessed for quality on the Agilent TapeStation (Agilent Technologies, Inc., Wilmington DE), and quantitative RT-PCR for library quantification was performed using the Kapa Library Quantification Kit (Roche Sequencing). The library was sequenced on an Illumina NextSeq 500 mid-output sequencing run to generate 125 million pairs of 75-base reads.

### (c) Patients 3-6

WES was performed on DNA from frozen tumor tissues and from blood as germline controls in order to identify somatic mutations in the coding regions of the human genome. Briefly, 200 ng of DNA was used as input into the Agilent SureSelect XT Clinical Research Exome kit (Agilent Technologies). For each tumor DNA sample, a genomic DNA library was constructed according to the manufacturer's protocol and the size and quality of the library was evaluated using the Agilent BioAnalyzer. An equimolar amount of library DNA was used for a whole-exome enrichment using the Agilent capture baits and after qPCR library quantitation and QC analysis on the BioAnalyzer, approximately 110 million and 60 million 75-base paired-end sequences for the tumor and germline control samples, respectively, were generated using v2 chemistry on an Illumina NextSeq 500 high-output sequencing run (Illumina, Inc.).

An RNAseq library was prepared using the Universal RNA-Seq Library Preparation Kit with NuQuant, (Tecan US, Inc.). Briefly, 100 ng of DNase-treated RNA was used to generate cDNA and a strand-specific library following the manufacturer's protocol. Library molecules containing ribosomal RNA sequences were depleted using the NuGen AnyDeplete probe-based enzymatic process. The final library was assessed for quality on the Agilent TapeStation (Agilent Technologies, Inc.), and quantitative RT-PCR for library quantification was performed using the Kapa Library Quantification Kit (Roche Sequencing). The library was sequenced on an Illumina NextSeq 500 mid-output sequencing run to generate >80 million pairs of 75-base reads per sample.

### (4) Neoantigen Peptide Detection and MHC Binding Analysis

Whole exome sequencing (WES) data from matched tumor and germline specimens were aligned to human genome version hs37d5 with the Burrows Wheeler Aligner (BWA) algorithm (v0.5.9-r16) and refined with Picard (v1.56, http://picard.sourceforge.net/) and the Genome Analysis Toolkit (GATK2Lite-2.2). Somatic point mutations were detected with MuTect (v1.14), SomaticIndelDetector (via GATK2Lite), and Strelka (v1.0.13) (Illumina, Inc.) analysis tools and annotated with ANNOVAR software. Somatic mutations observed as passing in Strelka, or passing in MuTect and observed at all in Strelka were used to predict altered peptides from the ANNOVAR results. RNA sequencing (RNASeq) data from tumor specimens were aligned via the Spliced Transcripts Alignment to a Reference (STAR, v2.5.3a) algorithm and quantitated by the High-Throughput Sequence Analysis tool (v0.6.0) (HTSeq) to confirm gene expression and verify mutation identification.

Each patient's complete HLA haplotype was predicted by RNASeq analysis via the Optitype (HLA-I, v1.3.2) and PHLAT (HLA_IL, v1.1) algorithms. This HLA profile was utilized to predict the binding of the mutated peptides to MHC Class I and MHC Class II components by netMHCpan (v4.0) and netMHCIIpan (v3.2), respectively. Mutant and wildtype peptides were extracted from the ANNOVAR output and binding peptide sizes of 8-14 amino acids (MHC-I) and 12-16 amino acids (MHC-2) were tested.

### (5) Neoantigen Peptide Prioritization

Mutated peptides were prioritized based upon expression and MHC binding prediction analyses to obtain an Additive Score (AS). The expression score (ES) component was determined using the maximum variant allele frequency in the RNAseq data (VAF) and fragments per kilobase of exon per million mapped fragments (FPKM) from the RNASeq data. The MHC combined score (MCS) component added the maximum predicted binding of each peptide to the patient's MHC molecules and the differential agretopicity index (DAI) between the variant (var) peptide and its corresponding reference (ref) peptide for MHC Class I (mhc1) and MHC Class II (mhc2). The full AS formula for prioritizing 25-mers is below:

$$AS = ES + ( MCS / 1.5 ) \\ ES = max\_VAF \_RNA \_percentile + ( 0.5 * max\_FPKM \_percentile ) \\ MCS = mhc1\_score + ( 0.5 * \$mhc2\_score ) \\ mhc1\_score = ic50\_mhc1 \_percentile + ( 0.5 * DAI\_mhc1 \_percentile ) \\ mhc2\_score = ic50\_mhc2 \_percentile + ( 0.5 * DAI\_mhc2 \_percentile ) \\ percentile = max percentile for a given peptide within 25 - mer ( excluding 0 / NA ) \\ DAI : var\_ic50 / ref\_ic50$$

### (6) Custom Peptide Synthesis

Prioritized peptides were synthesized individually as 25mers utilizing the PEPotec Immuno Custom Library (ThermoFisher Scientific) and Custom Peptide Synthesis (JPT Peptide Technologies, Berlin, Germany) platforms or as a peptide pool (PP) via the PepMix™ Peptide Pools platform (JPT Peptide Technologies). For Patient 1, an additional custom library of overlapping 12-16mer peptides harboring the S100A11^{Q22R} mutation were synthesized as well as the wildtype 25mer (WT; S100A11^{WT}) via the PEPotec Immuno Custom Peptide Library platform (ThermoFisher Scientific). All peptides were reconstituted in dimethyl sulfoxide (DMSO) and used at the indicated concentrations.

### (7) APC Generation

Autologous dendritic cells (DC) were derived from cryopreserved mononuclear cells from patient apheresis specimens collected as part of the clinical protocol prior to pre-conditioning lymphodepletion. Briefly, cells were thawed in warm RPMI-based media containing 10% human Ab serum (Lot 20800: Omega Scientific, Tarzana, CA; Lot H16Y00K: Gemini Bio Products, West Sacramento, CA; Lot A14006: Access Biologicals, LLC, Vista, CA) and 6.7 ug/mL DNase I (MilliporeSigma, St. Louis, MO). All cells were plated at 37° C. at a concentration of 1×10⁷/mL in DC Media (CellGenix, Inc., Portsmouth, NH). All non-adherent cells and media were removed after 90 minutes and replaced with fresh DC Media supplemented with 100 ng/mL GM-CSF (R&D Systems, Minneapolis, MN) and 20 ng/mL IL-4 (Miltenyi Biotec). Three days later, an equivalent volume of this same media was added. On day five, DC Media was removed and DC were washed with warm PBS and collected in warm PBS-EDTA (Lonza Group AG, Basel, Switzerland) using a cell scraper to detach adherent cells.

Autologous B cells were transformed from peripheral blood lymphocytes (PBL) utilizing Epstein-Barr Virus (EBV) supernatants produced by the B95-8 cell line (a kind gift from Dr. Ken Wright, MCC), according to established protocols. Briefly, 1×10⁷ PBL were added to an upright T25 flask in 5 mL of tumor complete media (TCM), 5 mL of EBV supernatant, and 0.5 ug/mL of cyclosporin A (ThermoFisher Scientific). TCM consisted of RPMI containing 10% fetal bovine serum (FBS; Atlanta Biologicals, Flowery Branch, GA). B cells were incubated until macroscopic clusters were observed and propagated until sufficient numbers were generated for downstream applications. Flow cytometric analysis was utilized to confirm population purity and expression of canonical B cell markers CD19 and CD20 (panel below).

### (8) TIL Neoantigen Peptide Stimulation

Autologous APCs were cultured at 3.6×10⁶/well in six-well plates and loaded with the complete pool of patient-specific mutant 25mer peptides (2.5-100 ng/mL each) at equal concentrations for approximately 24 hours in DC Media (CellGenix, Inc.). APCs were washed three times in PBS to remove unbound peptide and replated in six-well plate format for the co-culture. TIL, rested overnight in RPMI supplemented with 10% human Ab serum (TIL CM) and 3000 IU/mL recombinant human (rh) IL-2 (aldesleukin, Prometheus Laboratories, Inc., San Diego, CA), were enumerated and added to the peptide-loaded APCs at a 1:1 ratio for overnight co-culture. Co-culture media was supplemented with 300 IU/mL rhIL-2 to promote TIL viability. Cells were collected and stained for fluorescence-activated cell sorting (FACS) under sterile conditions according to the below procedure. Cell culture supernatants were assayed individually for IFNg by the Single Plex immunoassay or simultaneously for IFNg, TNFa, and Granzyme B using the multianalyte immunoassay and analyzed on the Ella machine (Protein Simple, San Jose, CA).

### (9) Fluorescence-Activated Cell Sorting (FACS)

All cells were collected and washed in sterile PBS, then stained with the Live/Dead Fixable Near-IR Dead Cell Stain Kit (ThermoFisher Scientific) according to the manufacturer's protocol at 1×10⁷ cells/mL. Cells were then washed with sterile Flow Cytometry Buffer (FCB) and stained with surface antibodies. FCB consisted of 5% FBS, 1 mM EDTA, and 0.1% sodium azide in PBS. Anti-human cell surface antibodies were added according to the following panel: CD11c BV650 (clone B-ly6; BD Biosciences, San Jose, CA), CD3 BV785 (clone UCHT; BioLegend, Inc., San Diego, CA), CD4 FITC (clone RPA-T4; BD Biosciences), CD8 BV510 (clone SKI; BioLegend), CD134 (OX40) BV421 (clone BerACT-35: BioLegend), CD137 (4-1BB) PerCyCy5.5 (clone 4B4-1; BioLegend). B cells were utilized as APCs when DCs were unavailable. In this event, CD20 BV650 (BD Biosciences) replaced CD11c BV650 in the sort staining panel. Cells were stained at 4° C. for 20 minutes, then washed with sterile FCB and resuspended in Complete OpTmizer Media for FACS. Complete OpTmizer Media consisted of CTS™ OpTmizer™ T cell Expansion SFM (no phenol red; ThermoFisher Scientific) supplemented with CTS™ Immune cell SR (ThermoFisher Scientific), GlutaMAX Supplement (ThermoFisher Scientific) and 300 IU/mL rhIL-2. TIL were sorted at a concentration of 5×10⁶-1×10⁷ cells/mL in Complete OpTmizer Media.

Neoantigen-specific TIL were sorted on a BD FACSAria SORP (BD BioSciences) for increased cell surface expression of 4-1BB and OX40 between peptide-stimulated TIL and unloaded controls. Additional control wells were set up in a 96-well format with 1×10⁵ TIL to assist with gating: (1) fluorescence minus one (FMO) staining for 4-1BB on TIL co-cultured with 1×10⁵ peptide-loaded APCs; (2) FMO for OX40 on TIL co-cultured with 1×10⁵ peptide-loaded APCs; (3) Dynabeads human T-activator CD3/CD28 (ThermoFisher Scientific) at a 1:1 TIL:bead ratio; (4) TIL cultured without APCs in media only. TIL activated by neoantigen peptides upregulated either 4-1BB or OX40 and were sorted as ‘positive’ while TIL that did not increase expression of either marker were sorted as ‘negative.’ TIL were sorted directly into human Ab serum supplemented with gentamicin, then washed twice with TIL CM. TIL were expanded via REP prior to validation and downstream experiments.

### (10) MHC Class II Expression

AT was induced to express MHC Class II by 1) pre-treatment with recombinant human (rh) IFNg (ATg) or 2) transduction with the Class II Transactivator (AT-CIITA). 1) Recombinant human IFNg (500 U/mL; R&D Systems) was added to TCM and cells were cultured for five to seven days total, with media replacement on day three. 2) AT was transduced utilizing CIITA viral supernatant supplemented with 4 ug/mL polybrene (Sigma-Aldrich, St. Louis, MO). Tumor cells were centrifuged with viral supernatant at 800 relative centrifugal force (rcf) for 90 minutes at 37° C., then expanded in TCM prior to puromycin (1 ug/mL; Sigma-Aldrich) selection. MHC Class II expression was confirmed by flow cytometry.

### (11) TIL Co-Culture

Effector (E) and target (T) cells were co-cultured at a 1:1 E:T ratio in a 96-well plate format with 1×10⁵ cells each. Effector cells consisted of TIL or TCR-T (below). Target cells consisted of AT, HLA-matched tumor, or EBV-transformed B cells. AT was enforced to express MHC Class II where indicated as described above. Target cells were loaded with individual peptides or a pool of peptides (2.5-150 ng/mL each) overnight prior to co-culture. Blocking antibodies were added one hour prior to co-culture initiation in order to pre-coat target cells and remained in the assay well for its duration at a final concentration of 10 ug/mL. Anti-human blocking antibodies utilized: HLA-ABC (clone W6/32; BioLegend, Inc.), HLA-DR, DP, DQ (clone Tu39; BioLegend, Inc.), HLA-DR (clone L243; BioLegend, Inc.), HLA-DP (clone B7/21; Leinco Technologies, Fenton, MO), HLA-DQ (clone SPV-L3; Novus Biologicals, Littleton, CO). Cell culture supernatants were collected and IFNg production was quantified by IFNg ELISA (R&D Systems) or the Ella system (described above).

### (12) Flow Cytometry

Following co-culture in a U-bottom 96-well plate format, TIL were transferred to a V-bottom 96-well plate, washed with PBS and stained with Live/Dead Fixable Near-IR Dead Cell Stain Kit (ThermoFisher Scientific) according to the manufacturer's protocol. TIL were then washed with FCB and stained with cell surface antibodies for 30 minutes at 4 C, protected from light. Cells were washed with FCB and fixed. For surface staining, TIL were fixed in 2% paraformaldehyde (PFA) until acquisition.

For intracellular cytokine staining (ICS), GolgiStop and GolgiPlug (BD Biosciences) were added at 0.5× according to the manufacturer's recommended dosage at the initiation of co-culture. For degranulation potential, CD107a (LAMP-1) BV785 (clone H4A3; BioLegend, Inc.) was also added at co-culture initiation. TIL were viability and surface stained as above, then processed and stained with the Fixation/Permeabilization Solution Kit (BD Biosciences) according to the manufacturer's protocol and fixed in 2% PFA until data acquisition. Data acquisition was performed on a BD Celesta or BD LSRII (BD Biosciences) and analyzed with FlowJo Software (TreeStar Inc., Ashland, OR).

### (13) Flow Cytometry Antibodies

The following anti-human antibodies were utilized for flow cytometry as described above: CD3 BV785 (clone UCHT1; BioLegend, Inc.), CD4 FITC (clone RPA-T4; BD Biosciences), CD8 BV510 (clone SKI; BioLegend, Inc.), CD11c BV650 (clone B-1y6; BD Biosciences), CD20 BV650 (clone 2H7; BD Biosciences), CD25 PECy7 (clone M-A251; BD Biosciences), CD39 BV605 (clone A1; BioLegend, Inc.), CD69 BV510 (clone FN50; BioLegend, Inc.), CD107a (LAMP-1) BV785 (clone H4A3; BioLegend, Inc.), CD127 APC (clone A019D5; BioLegend, Inc.), CD134 (OX40) BV421 (clone Ber-ACT35; BioLegend, Inc.), CD137 (4-1BB) PerCpCy5.5 (clone 4B4-1; BioLegend, Inc.), CD183 (CXCR3) BV421 (clone 1C6; BD Biosciences), CD223 (LAG-3) BV650 (clone 11C3C65; BioLegend, Inc.), CD279 (PD-1) BV785 (clone EH12.2H7; BioLegend, Inc.), CD366 (TIM3) APC (clone F38-2E2; eBiosciences), BTLA BV650 (clone J168-540; BD Biosciences), Granzyme B PacBlue (clone GB11; BioLegend, Inc.), IFNg APC (clone 4S.B3; eBiosciences), TNFa PECy7 (clone Mab11; eBiosciences).

Infused TIL products utilized in clinical trial protocols were cell surface stained and analyzed to determine CD4 and CD8 frequency in the TIL product.

### (14) TCRβ Sequencing

DNA was extracted from TIL and PBMC via the DNeasy Blood and Tissue Kit or AllPrep DNA/RNA Mini Kit (Qiagen Sciences, Inc.) and quantified by Nanodrop (ThermoFisher Scientific). When necessary, DNA was precipitated using isopropanol and re-quantified. DNA was analyzed using the ImmunoSeq TCRb Kit v3 and v4 (Adaptive Biotechnologies, Seattle, WA) at the MCC Molecular Genomics Core or the Adaptive Biotechnologies ImmunoSeq Laboratory. Data were uploaded to the Adaptive ImmunoSEQ Analyzer 3.0 for analysis of shared TCRb CDR3 amino acid sequences.

### (15) Incucyte Live Cell Imaging

Autologous EBV-transformed B cells (targets) were pulsed with 100-150 ng/mL of peptide overnight. Target cells were labeled with 0.2 uM Incucyte Cytolight Rapid Green Dye (Essen Bioscience, Inc., Ann Arbor, MI) according to the manufacturer's protocol, washed, and plated in Complete OpTmizer Media at 2×10⁴ Target cells/well in a flat bottom 96-well plate. The assay plate was pre-coated with poly-L-ornithine (Sigma-Aldrich) for 60 minutes at room temperature, then allowed to air dry for 30 minutes at room temperature. Peptides (100-150 ng/mL) and IL-2 (300 IU/mL) were included for the duration of the assay. The Incucyte Caspase 3/7 Red Dye (Essen Biosciences, Inc.) was resuspended in Complete OpTmizer Media and added to each well at a final concentration of 2 uM/well. TIL (effectors) were rested overnight in TIL CM supplemented with 3000 IU/mL rhIL-2, then counted and added to the assay plate at 1:1, 2.5:1, 5:1, and 10:1 E:T ratios. Co-culture plates were immediately placed in the Incucyte S3 Live Cell Analysis Instrument (Essen Biosciences Inc.) and images were acquired at 30-minute intervals for six hours. Data analysis was completed with the Incucyte S3 Software v2018B (Essen Biosciences Inc.) and exported to GraphPad Prism v9 (GraphPad Software, La Jolla, CA).

### (16) Real-Time Cell Analysis (RTCA) Immune Cell Killing Assay

AT cell targets were collected with 0.05% trypsin (ThermoFisher Scientific), enumerated, and plated on the E-Plate 96 PET (Agilent Technologies) in TIL CM at a concentration of 1×10⁵ cells/well. Each AT-bearing plate was initially calibrated on the xCELLigence RTCA MP instrument (Agilent Technologies), then incubated for approximately 24 hours within the analyzer chamber. Enriched TIL (effectors) were added to the plate at 1:1, 2.5:1, 5:1, and 10:1 E:T ratios in triplicate and returned to the RTCA analyzer chamber for six hours. Plate sweeps occurred at 15-minute intervals and measured cellular impedance of AT in real-time during each step. Maximum target cell death was achieved via addition of 2% Triton X-100 (MilliporeSigma) in lieu of effector cells. Data was acquired and analyzed with the RTCA Software Pro Immunotherapy Module (Agilent Technologies) and exported for analysis and visualization with GraphPad Prism v9 (GraphPad Software) for data visualization.

### (17) Transgenic T Cell Receptor-Engineered T Cell (TCR-T) Production

Full-length T cell receptor (TCR) alpha and beta chain sequences were extracted from scRNASeq VDJ analysis and synthesized as double-stranded DNA fragments (gBlock; Integrated DNA Technologies, Inc., Coralville, IA). The TCR sequences were arranged in tandem, linked by the porcine teschovirus-1 (P2A) self-cleaving peptide sequence, and flanked by restriction enzyme sites (NcoI and NotI) to create the insert sequence. The insert and plasmid backbone (MSGV1) were digested, purified, and ligated. The full TCR plasmid was transformed into One Shot Stbl3 chemically-competent E. coli (ThermoFisher Scientific) and amplified on LB agar plates containing ampicillin (100 ug/mL) for purification by the QIAGEN Plasmid Maxi Kit (Qiagen Sciences, Inc.) according to the manufacturer's protocol. The TCR plasmid and envelope protein plasmid (RD114) were co-transfected into 293GP cells utilizing Lipofectamine 2000 (ThermoFisher Scientific). Retroviral supernatants were collected at 48-, 72-, and 96-hours following transfection, filtered at 0.45 microns, and utilized for TCR-T transduction.

Allogeneic peripheral blood lymphocytes (PBL) were isolated from whole blood via Ficoll-Hypaque density gradient (MP Biomedicals, Santa Ana, CA) and red blood cells lysed with ACK lysis buffer (ThermoFisher Scientific). T cells were activated with 0.25 ug/mL of anti-CD3 (clone: OKT3; BioLegend, Inc.) for 48 hours in X-VIVO media (Lonza Group AG) supplemented with 5% HS (Gemini Bio-Products, Inc., Sacramento, CA), 1% L-glutamine (ThermoFisher Scientific), and 300 IU/mL IL-2 (Proleukin, Clinigen Group, Burton upon Trent, UK). Six-well plates were pre-coated with 20 ug/mL RetroNectin (Takara Bio, Kusatsu, Shiga, Japan), then blocked with PBS supplemented with 2% bovine serum albumin (BSA; MilliporeSigma). After rinsing with PBS, 2 mL of viral supernatant was added to the RetroNectin-coated plates with an equal volume of DMEM supplemented with 10% FBS. Plates were centrifuged at 2000 rcf for two hours at 32C. Activated ap T cells were transferred to viral-coated plates at 2e6 T cells/well and centrifuged for ten minutes at 1000 rcf at 32° C., then incubated at 37° C. overnight. This transduction procedure was repeated after 24 hours in a duplicate set of pre-coated six-well plates as above. Following transduction, the TCR-T cells were transferred to flasks and expanded for 72 hours in X-VIVO media supplemented with 300 IU/mL IL-2. TCR-T cells were evaluated for transduction efficiency by flow cytometric detection of the mTCRb construct (anti-mouse TCRbeta PerCP/Cy5.5, clone H57-597; BioLegend, Inc.) on viable CD4⁺ (anti-human CD4 PECy7, clone RPA-T4; BioLegend, Inc.) and CD8⁺ lymphocytes (anti-human CD8 BUV395 RPA-T8; BD Biosciences) and utilized in functional assays.

(18) Single Cell RNA Sequencing (scRNASeq)

Sorted TIL from six melanoma patients were cryopreserved following REP and scRNASeq was performed by Turnstone Biologics immediately upon thaw. TIL were counted via the Countess Automated Cell Counter (ThermoFisher Scientific) with trypan blue, filtered, and resuspended between 700-1300 cells/uL in order to sequence 2000 cells per sample. The 15 Chromium Next GEM Single Cell 5′ Reagent Kits v2 (Dual Index) (10× Genomics, Pleasanton, CA) was used for paired TCR- and scRNA-sequencing following the manufacturer's protocol. The Chromium Next GEM Chip K was loaded with gel beads in emulsion (GEM) containing T cells, master mix components, barcoded gel beads, and partitioning oil. Reverse transcription generated barcoded cDNA and V(D)J sequences for amplification, followed by 5′ Gene Expression (GEX) and V(D)J library construction. Sequencing was performed on the NextSeq 1000 Instrument (Illumina) at a depth of 60 million (GEX) and 30 million (V(D)J) paired-end reads, per sample.

### (19) Single-Cell RNA-Seq Data Processing, Filtering, Batch Effect Correction, and Clustering

Raw sequencing reads for each sample were processed using Cell Ranger (v6.1, 10λ Genomics) and aligned against GRCh38 human transcriptome. Barcodes with unique molecular identifier (UMI) counts passing threshold were imported to Seurat v4.2.0 for further analysis. Genes detected in less than 3 cells were excluded; cells with less than 200 genes detected or greater than 10% mitochondrial UMIs were filtered out. Doublets were detected using Scrublet, DoubletFinder, scDblFinder, and doubletCells implemented in scran, assuming 0.08% doublet rate for every 1000 sequenced cells. Cells identified as doublets by at least two algorithms were removed from further analysis. UMI counts were log normalized. The top 5000 variable genes were identified using FindVaribleFeatures function with “vst” method. T cell receptor and immunoglobulin genes were removed from the variable genes to prevent clustering based on V(D)J transcripts. S and G2/M cell cycle phase scores were assigned to cells using CellCycleScoring function. Top 40 principal components were generated by RunPCA and further used to construct the uniform manifold approximation and projection (UMAP) by RunUMAP. Clusters were identified by Louvain algorithm using FindCluster at resolution=1. For each sample, the CD8⁺ and CD4⁺ T cells were identified based on expression of CD3E, CD3D, CD4, CD8A, and CD8B, as well as clustering results as following: 1) CD4⁺CD8A⁻CD8B⁻ cells were assigned as CD4⁺ cells; 2) CD4⁻CD8A⁺/CD8B⁺ cells were assigned as CD8⁺ cells; 3) For CD4⁺CD8A⁺/CD8B⁺ cells, the cells with log2(CD4/CD8) >1 and clustered with CD4⁺ cells were assigned as CD4⁺, while the cells with log2(CD4/CD8)<1 and clustered with CD8⁺ cells were assigned as CD8⁺.

The CD4⁺ cells from individual samples were further integrated to remove batch effects using FindintegrationAnchors and IntegrateData functions with 8000 anchor genes and 40 dimensions of canonical correlation analysis (CCA). Integrated data were regressed against total UMIs, percentage (%) of mitochondrial UMIs, and cell cycle phase scores using ScaleData. A shared nearest neighbor (SNN) based graph was constructed using top 40 principal components, and clusters were identified by Louvain algorithm using FindCluster at resolution=1. UMAP projections were generated by RunUMAP and used for all visualizations.

### (20) Differential Gene Expression Analysis and Cluster Annotation

Differential expression analysis comparing each cluster versus all others was performed using FindAllMarkers function in Seurat with default settings. Genes with log2(fold-change)>0.25 and Bonferroni-corrected p-value <0.05 were considered differentially expressed. Clusters were annotated by comparing differential genes to markers in scRNA-seq studies. Clusters were merged into cell types based on above annotation. Enrichment scores of the T cell exhaustion gene set (TOX, LAG3, PDCD1, HAVCR2, ITGAE, TIGIT, CXCL13) and the T cell stemness gene set (TCF7, CDCR5, CD28, GZMK, CCR7, IL7R, BCL6, SELL, CD27) were calculated using AUCell algorithm implemented in SCENIC. Cells localized to the ‘proliferative’ cluster were present across all patient samples and excluded from the analysis. Marker genes expression was visualized on the UMAP or by violin plot using log-normalized UMI counts. A bubble plot was used to visualize z-score normalized average expression and percentage of expressing cells per cluster or per cell type. Expression distribution of marker genes were compared between cell types using Violin plots.

### (21) Gene Set Enrichment Analysis (GSEA)

Differential expression analysis was performed comparing cells within each cell type versus all other cells, followed by a gene set enrichment analysis (GSEA). For each cell type, genes were ranked based on −log10(p-value)*(sign of log2(fold-change)) resulted from the differential analysis, with the most up-regulated genes at top and the most down-regulated genes at bottom. Pre-ranked GSEA was performed on gene rankings using R package fgsea with 10,000 permutations, against Hallmarks, KEGG, BIOCARTA, REACTOME, PID, Gene Ontology, and ImmuneSigDB databases from MsigDB. The normalized enrichment scores (NES) were visualized using GraphPad Prism for selected pathways. Full GSEA is reported in the Supplemental Data Files.

### (22) Single-Cell 10× V(D)J Analysis

TCR reads sequenced by 10× V(D)J assay were aligned to human GRCh38 reference transcriptome using Cell Ranger VDJ (v6.1, 10× Genomics) to assemble the single TCR chains. Only the assembled chains that were highly confident, of full-length, and productive were kept for downstream analysis. Cells with the same amino acid sequences of the CDR3 regions and V(D)J genes for both TRA and TRB chains were considered originated from the same clone. These cells were further assigned to cell types based on their annotation of the paired single-cell RNA assay. Identified TCRs were matched to the known sequences of neoantigen specific TCRs in each patient. Cells expressing neoantigen specific TCRs were visualized on UMAP projected generated from paired RNA assay.

### (23) Statistical Analysis

All statistical analyses utilized GraphPad Prism v9 (GraphPad Software) with statistical methods reported in figure legends. Unless otherwise indicated, error bars represent mean and standard deviation (SD) of technical replicates.

### b) Results

### (1) Patient 1

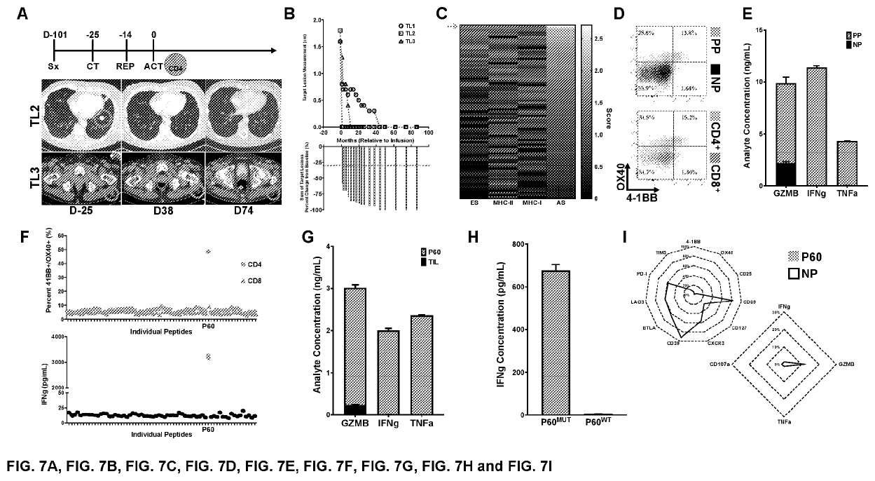

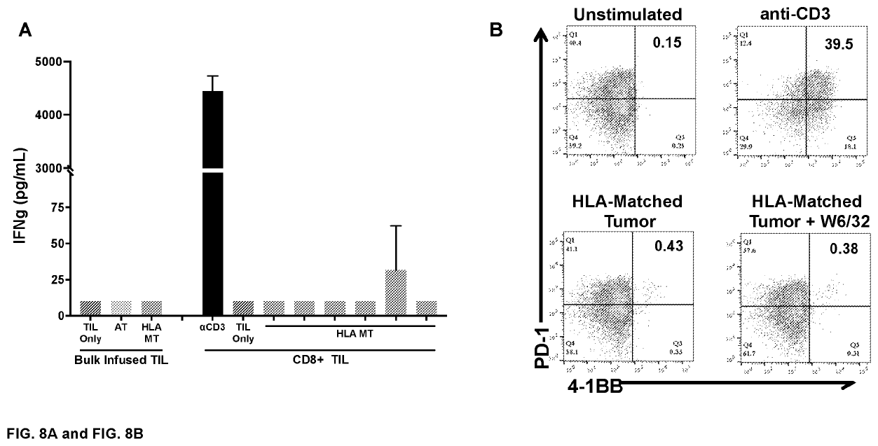

Patient 1 was a 49-year-old male who presented with M1c metastatic melanoma refractory to multiple treatments prior to ACT utilizing TIL. Surgical resection of an intramuscular arm lesion yielded 48 fragments for TIL generation, resulting in 5.2×10¹⁰ predominantly CD4⁺ TIL (88%) for infusion 101 days following surgery.² Multiple metastases were quickly resolved upon ACT, and the patient ultimately achieved an ongoing long-term complete response (CR) of greater than ten years (FIGS. 7A and 7B). Infused TIL failed to produce IFNg in vitro in response to autologous tumor (AT). As the infusion product contained a low proportion of CD8⁺ T cells, we hypothesized that enriching this fraction would uncover tumor specificity given the observed tumor regression following TIL transfer. However, co-culture with a panel of HLA-matched melanoma tumors still did not elicit IFNg release by the CD8⁺ TIL (FIGS. 8A and 8B). As standard methods for tumor reactivity by CD8⁺ T cells did not seem to reflect the overwhelming clinical response, we posited that neoantigen recognition by CD4⁺ T cells drive the anti-tumor response in vivo.

To investigate this, we performed whole exome (WES) and RNA sequencing (RNASeq) from surgical tumor tissue in order to determine this patient's neoantigen repertoire. In total, 91 non-synonymous mutations were detected and 79 of these mutations were scored by expression level and predicted binding affinity to the patient's specific MHC haplotype (FIG. 7C). Of these, 65 peptides were amenable to custom synthesis. When pooled and loaded onto autologous DCs, efficient stimulation of the TIL infusion product was detected via upregulation of OX40 and 4-1BB, markers of antigen-specific T cell activation. This response was confined to the CD4⁺ TIL fraction, with minimal stimulation of the CD8+ T cells (FIG. 7D). Further evaluation indicated that the neoantigen peptide pool elicited a multifaceted effector response from these TIL, with the release of the cytolytic enzyme granzyme B, and the critical anti-tumor cytokines, IFNg and TNFa (FIG. 7E).

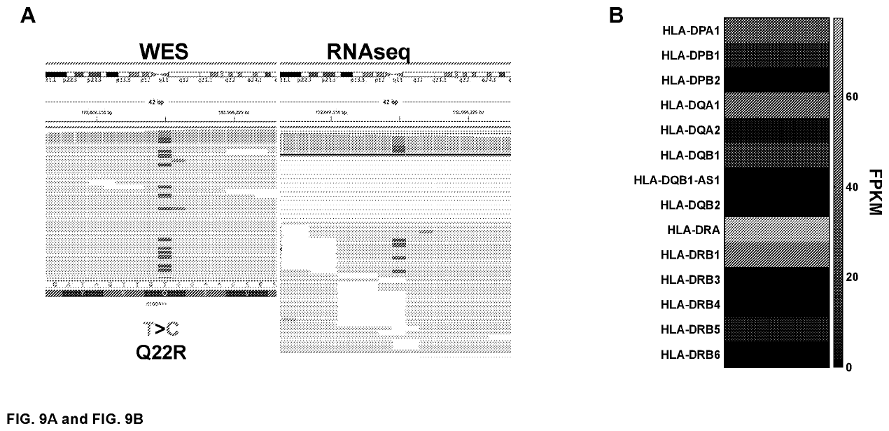

To determine precisely which neoantigens were driving the observed reactivity, we pulsed each mutated peptide individually onto APCs, followed by co-culture with the infused TIL product. Only one of the 65 peptides (peptide 60, P60) produced significant signal from the TIL product in terms of co-stimulatory molecule induction and cytokine secretion. Again, this reactivity was produced exclusively by the CD4⁺ TIL compartment (FIG. 7F). Moreover, the complement of effector molecules released in response to P60 recapitulated the response to the pooled peptides (FIG. 7G). Peptide 60 was identified as a mutated variant of the S100A1 protein, which carried a Q22R amino acid conversion derived from a non-synonymous T>C substitution. WES and RNASeq confirmed substantial expression of this antigen in approximately 40% of the sequenced reads from the patient's tumor tissue (FIG. 9A). Further, S100A11^{Q22R} ranked as the highest prioritized neoantigen from this patient (FIG. 7C).

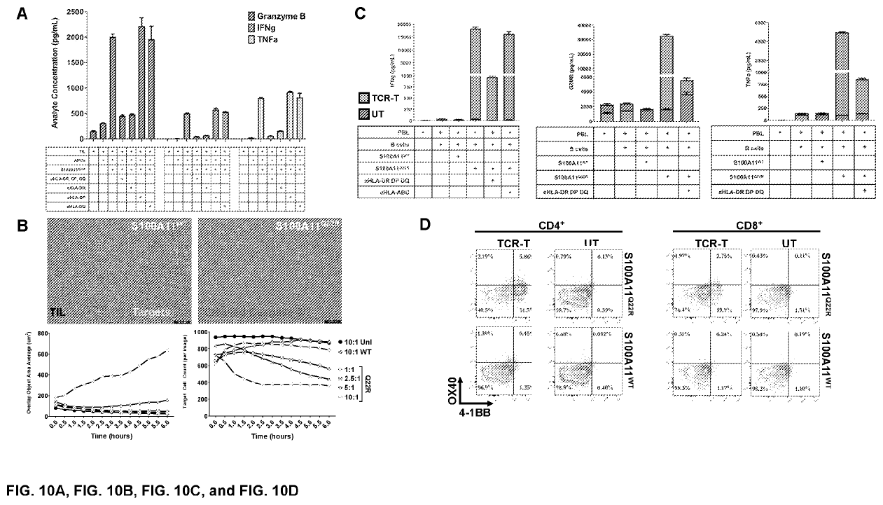

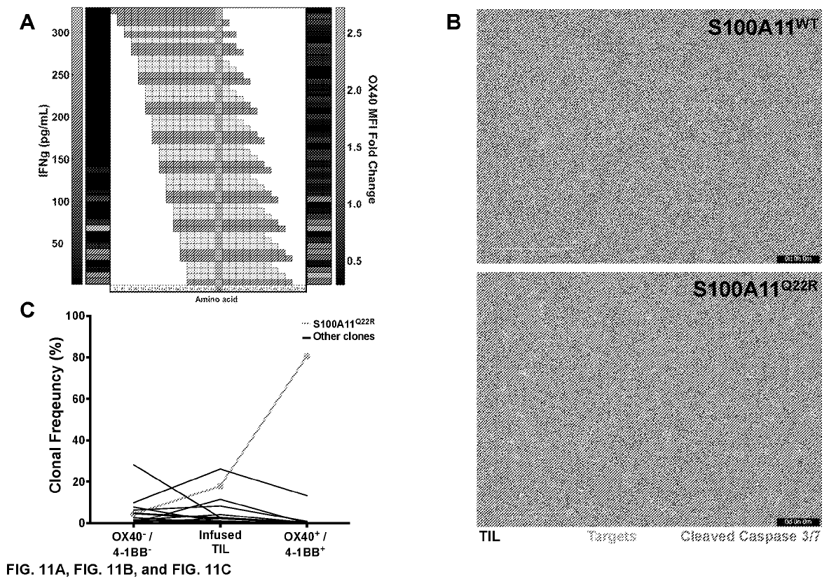

We synthesized the cognate wildtype (WT) peptide to ensure that these CD4⁺ T cells were specific for the mutated S100A11^{Q22R} peptide. Patient 1's TIL did not produce IFNg in response to WT peptide, indicating specific recognition of the mutated version of this neoantigen (FIG. 7H). Further interrogation by flow cytometry revealed that the CD4⁺ TIL response to the S100A11^{Q22R} peptide induced upregulation of additional surface markers of activation, including LAG-3, PD-1, TIM3, CD25, and CD69, while expression of CD127 and CXCR3 were reduced. Intracellular staining revealed substantial production of Granzyme B, IFNg, TNFa, and CD107a. indicative of functional degranulation and effector cytokine production (FIG. 7I). Neoantigen-directed cytokine release was abrogated when target cells were pre-coated with either a pan-MHC Class II or HLA-DR antibody, but not with an HLA-DP or HLA-DQ antibody (FIG. 10A). RNA expression of the HLA-DR subunits in preserved tumor tissue supported neoantigen presentation by HLA-DR as a mechanism for anti-tumor recognition by CD4⁺ TIL (FIG. 9B). Minimal epitope screening of 12- to 16mer peptides spanning the full 25mer mutant peptide sequence indicated a critical recognition motif near the C-terminus (FIG. 11A).

To better understand the impact of CD4⁺ TIL recognition of aberrant S100A11 expression by targets, we performed an in vitro cytotoxicity assay. Utilizing live cell imaging, we found that CD4⁺ TIL rapidly clustered with target cells loaded with S100A11^{Q22R} peptide, indicating efficient recognition and interaction. Over the course of six hours, target cells pulsed with mutant peptide were eliminated in a dose-dependent manner, as determined by decreased target cell count and increased overlap of cleaved caspase 3/7 staining in target cells over time. These effects were not observed when targets were pulsed with WT S100A11 peptide, again demonstrating specificity of the CD4⁺ TIL response (FIG. 10B and FIG. 11B).

Neoantigen-reactive TIL were enriched through fluorescence-activated cell sorting (FACS) by upregulation of OX40 and 4-1BB (FIG. 1C). A single dominant clone (>80%) with paired TRAV6 and TRBV18 chains that recognized S100A11^{Q22R} was identified via TCRb and single cell TCR sequencing. TRC-T were engineered with the full-length TCR sequence and co-cultured with APCs bearing S100A11^{Q22R} and S100A11^{WT} peptide. The TCR-T produced high levels of effector cytokines (IFNg, TNFa, and Granzyme B), which was dependent on alignment of TCR, peptide, and MHC Class IL (FIG. 10C). Surface upregulation of 4-1BB, rather than OX40, governed this response for both CD4⁺ and CD8⁺ TCR-T (FIG. 10D). Despite CD8⁺ TCR-T activation, production of IFNg was not hindered by MHC Class I blockade, indicating that the CD8⁺ TCR-T cells directly recognized the peptide-MHC Class II complex independent of the CD8 co-receptor.

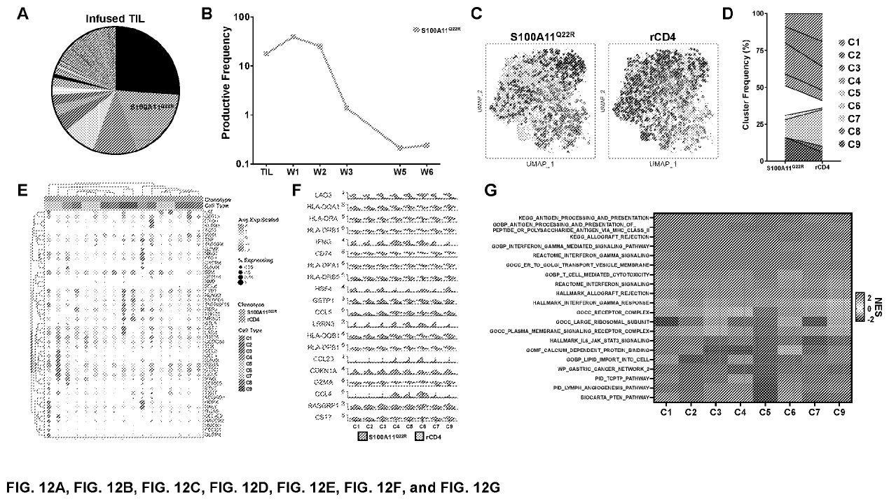

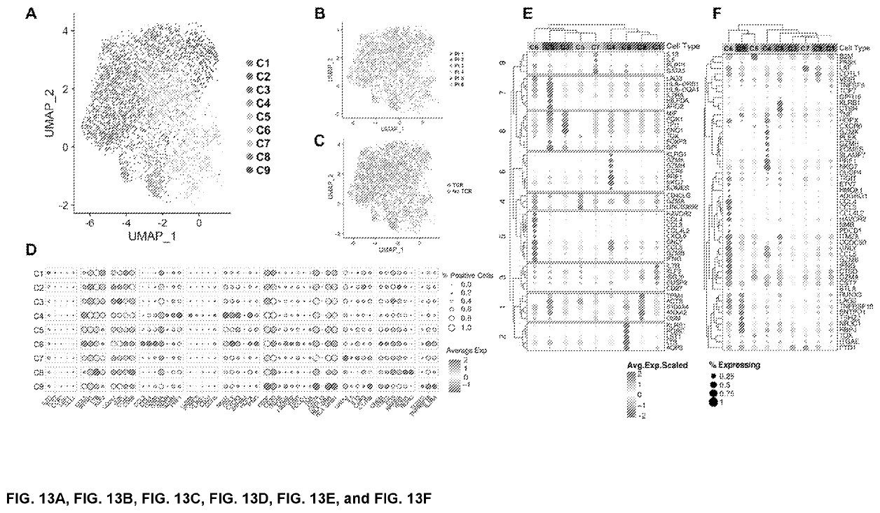

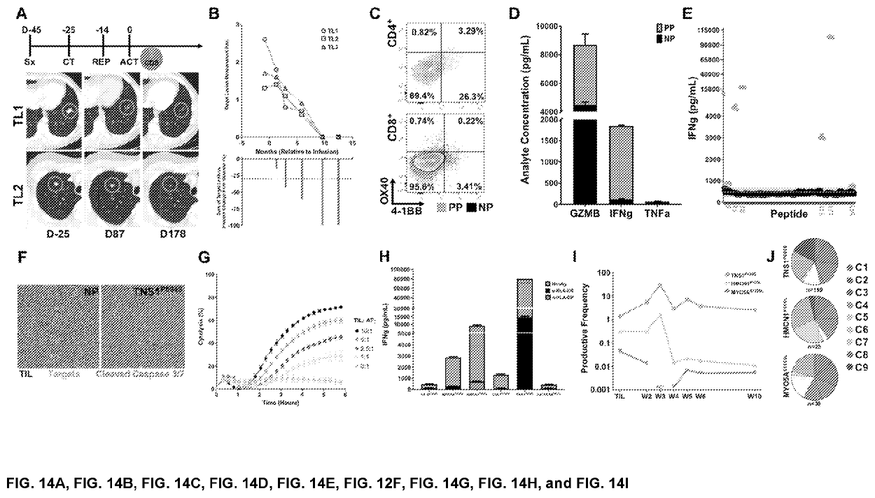

Utilizing the TCRb sequence, we tracked the CD4⁺ S100A11^{Q22R}-specific clone throughout longitudinal patient peripheral blood samples to understand the persistence and trajectory of this clone during the patient's anti-tumor response. At infusion, this clone comprised 17% of the bulk TIL product, the 2^{nd}-most abundant clone at the time of ACT (FIG. 12A). After one week (W1), this clone expanded to become the most prevalent clone, at nearly 40% of the peripheral repertoire. Of the top ten clones in the infusion product, this represented the largest fold increase immediately after infusion, indicating an initial clonal expansion. At W2, this clone maintained a 40% increase over its infusion frequency. After this time point, the endogenous T cell repertoire typically rebounds sharply from lymphodepletion, effectively diluting the frequency of adoptively transferred TIL.⁷³ Nevertheless, the S100A11^{Q22R}-specific clone remained in the top 50 clones (45^{th}) at least six weeks following infusion, indicating high persistence in the peripheral blood and indicating a sustained response to this neoantigen during the patient's clinical response (FIGS. 12B and 7B).

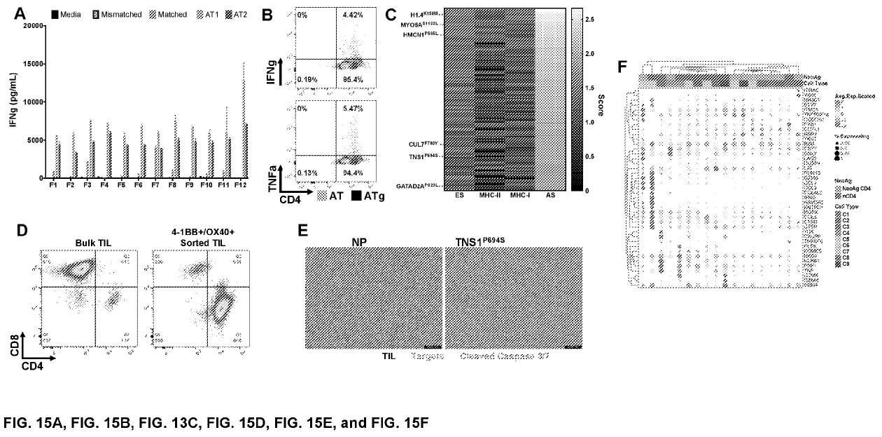

To further characterize this neoantigen-reactive CD4⁺ T cell clone and to better understand its relationship with other infused CD4⁺ TIL, tandem scRNASeq and TCRSeq were performed. Including Patient 1, the CD4⁺ TIL product from six metastatic melanoma patients were sequenced in this retrospective analysis, each previously treated with ACT on completed clinical trials at MCC. In total, 4,903 CD4⁺ T cells were sequenced followed by unsupervised clustering (FIG. 13A). Patient samples were distributed evenly across the uniform manifold approximation and projection (UMAP) and TCR expression was verified by VDJ recombination (FIGS. 13B and 13C). T cell clusters (C1-9) were annotated according to canonical gene modules and further characterized by the top differentially expressed genes (DEG) and genes that enriched in neoantigen-specific CD4⁺ T cells (FIGS. 13D, 13E, and 13F).

The S100A11^{Q22R}-specific CD4⁺ TIL clone was widely distributed across the UMAP clusters, with the exception of C8 indicating a lack of RORC+ Th17-like cells. The overall clonal profile was enriched for clusters C3, C5, and C9, consistent with T cells bearing an activated, cytotoxic and exhausted program, when compared to the residual CD4⁺ TIL (rCD4) from this patient (FIGS. 12C and 12D). The S100A11^{Q22R}-specific CD4⁺ TIL were expressed genes derived from neoantigen-specific CD4+ T cell signatures (FIG. 12E and FIG. 13F). Furthermore, this alignment with known markers of neoantigen reactivity was dictated primarily by the S100A11^{Q22R}-specific clone, rather than individual clusters, indicating high similarity between CD4⁺ T cells within this clone. The hallmark clonal genes included LAG3, JFNG, chemokines and MHC Class II molecules, which were significantly upregulated in comparison to rCD4 TIL (FIG. 12F). Gene set enrichment analysis (GSEA) revealed pathways involved in MHC Class II antigen processing and presentation, IFNg signaling, T cell cytotoxicity, and allograft rejection (FIG. 12G and Table 1). These modules were highly conserved across the clusters for the S100A11^{Q22R}-specific CD4⁺ TIL clone in comparison to rCD4. Together, these data support the S100A11^{Q22R}-reactive CD4⁺ TIL as a neoantigen specific, activated, effector T cell clone with cytotoxic capabilities, consistent with the functional activity of these CD4⁺ TIL.

### (2) Patient 2

To determine whether neoantigen-specific CD4⁺ TIL were present in other long-term responders to ACT with TIL, we investigated an additional metastatic melanoma patient treated with TIL. Patient 2's TIL product contained 88% CD8⁺ T cells and efficiently eradicated the patient's tumor burden following ACT, resulting in a CR and PFS of 60 months (FIGS. 14A and 14B). The bulk infusion product was highly reactive to AT in vitro; however further interrogation demonstrated evidence of tumor-reactive CD4⁺ TIL when pre-selected by IFNg secretion (FIGS. 15A and 15B).

Mutanome analysis of Patient 2's tumor identified 1631 non-synonymous mutations, of which 147 neoantigen peptides were synthesized and pulsed onto autologous APCs as a peptide pool (FIG. 15C). CD4⁺ TIL were preferentially activated as observed by 4-1BB and OX40 induction (FIG. 14C). As with Patient 1, this corresponded with Granzyme B, IFNg, and TNFa secretion (FIG. 14D). TIL from Patient 2 were sorted on upregulation of 4-1BB and OX40, which resulted in a dramatic population shift toward CD4′ TIL (NeoAg CD4) (FIG. 15D). When the NeoAg CD4 were screened against each individual mutant peptide, six neoantigen ‘hits’ stimulated a robust CD4⁺ T cell activation via IFNg release, indicating the presence of at least six neoantigen-reactive CD4⁺ TIL clones (FIG. 14E). Utilizing live cell imaging, NeoAg CD4 TIL rapidly formed T cell-target complexes in vitro, resulting in induction of cleaved caspase 3/7 and dose-dependent elimination of MHC-II⁺ AT cells (FIG. 14F, 14G, FIG. 15E). Each neoantigen response was efficiently abrogated in the presence of a pan-MHC Class II blocking antibody, five of which were found to be HLA-DR dependent (H1.4^{K156M} HMCN1^{P565L}, CUL7^{F780Y}, TNS1^{P694S}, GATAD2A^{P623L}), while one was HLA-DP dependent (MYO5A^{S1153L}) (FIG. 14H).

In order to deconvolute the precise TIL clone and neoantigen pairs, we sorted each neoantigen-specific CD4⁺ TIL clone individually. We focused on the top three sorted clones, which comprised greater than 90% of the sum frequency of the NeoAg CD4 TIL. The TNS1^{P694S}-specific clone represented the 8^{th}-most prevalent clone in the infusion product. Following infusion, this clone increased in relative frequency to become the 3^{rd}-ranked clone at W2 and peaked at W3 as the top clone in the periphery, representing over 30% of the T cell repertoire. The TNS1^{P694S}-specific clone continued to maintain relevance throughout the duration of the patient's response after this maximum, registering as the 3^{rd}-most prevalent clone in each subsequent peripheral blood draw through ten weeks. The HMCN1^{P565L}_specific clone was the 24^{th} ranked clone in the infusion product and similarly peaked at W3 as the 10^{th} most prevalent clone in the periphery, marked by a five-fold expansion in relative abundance. The MYO5A^{S1513L}-specific clone was the 120^{th} ranked clone at time of infusion, peaked at W2. and preserved similar relative abundance at W10 (FIG. 14I). Summarily, neoantigen-reactive CD4⁺ TIL clones persisted in the patient's periphery and demonstrated the ability to expand following infusion, indicating clonal amplification and maintenance in response to antigen recognition.

Clonal analysis by scRNASeq demonstrated a strong presence of C3, C4, and C5 in each of the three neoantigen-specific CD4⁺ TIL clones (range: 32-45%). These clusters were distinguished by genes indicative of activated effector and cytotoxic T cells, including PRF1, GZMA, GZMH, GZMK, and NKG7. As with Patient 1, a broad distribution of TIL from each identified NeoAg CD4 clone was observed, including notable C2 and C7 clusters. Comparing NeoAg CD4 to CD4⁺ TIL sorted simultaneously for the absence of OX40 or 4-1BB induction (nCD4), again revealed that NeoAg CD4 TIL were distinct from their counterparts and enriched for a subset of genes associated with neoantigen-specific CD4⁺ T cells (FIG. 15F). Cytotoxicity-associated genes were present in both NeoAg CD4 and nCD4, predominantly in cluster C6, indicating that additional CD4⁺ TIL capable of tumor cell lysis can also be present within this sample. Overall, these data supported our in vitro functional data demonstrating that the NeoAg CD4 TIL clones displayed an effector T cell profile marked by CD4-mediated AT-directed cytotoxicity.

### (3) Patients 3 and 4

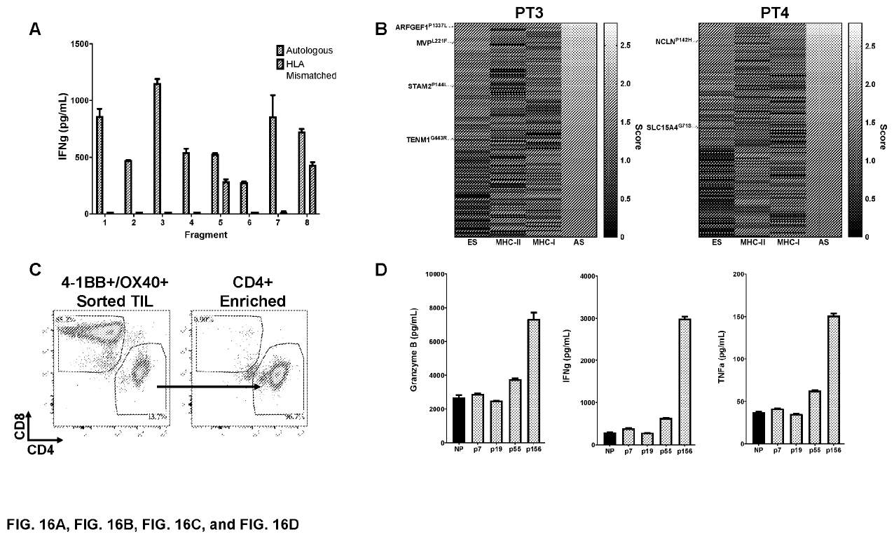

Next, we investigated whether neoantigen-reactive CD4⁺ TIL were present in the infusion product from patients who were non-responders to ACT with TIL. Patients 3 and 4 were each treated with predominantly tumor-reactive CD8⁺ TIL (97% and 58%, respectively) in the combination therapy setting (FIG. 16A). Patient 3 reached stable disease with a PFS of seven months, while Patient 4 had a PFS of 10 months. We again pursued neoantigen identification from WES and RNASeq and evaluated the infused TIL from Patient 3 and 4 for reactivity against 192 and 191 neoantigen peptides, respectively (FIG. 16B).

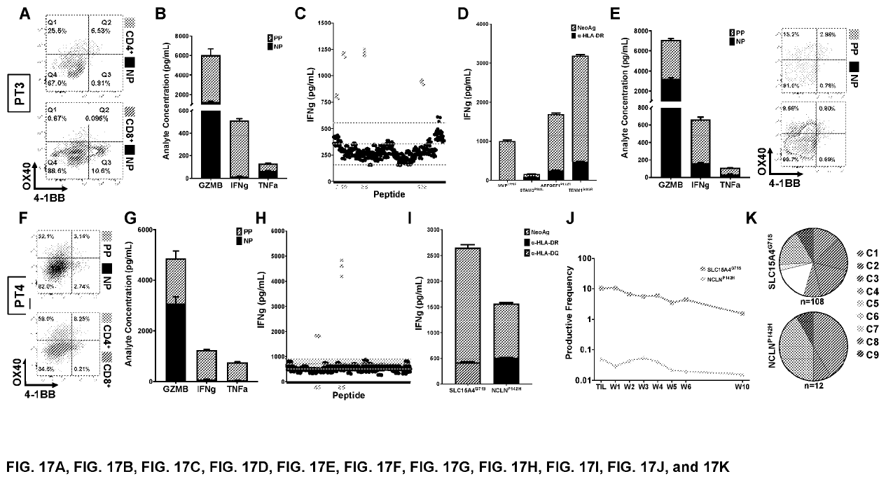

Patient 3's TIL product contained both CD4⁺ and CD8⁺ neoantigen-specific TIL, yet a greater proportion of the CD4⁺ T cells were activated in response to the mutant peptide pool (FIG. 17A). This corresponded with IFNγ, TNFα, and Granzyme B secretion, indicating the presence of potent effector T cells capable of a cytotoxic response (FIG. 17B). Individual peptide screening uncovered four mutated neoantigen hits that each induced IFNγ production from TIL (FIG. 17C). As CD4⁺ T cells were in the minority of the TIL product, we enriched for CD4⁺ TIL for further analysis (FIG. 16C). Following CD4-enrichment, reactivity was maintained in response to three of the four neoantigen peptide hits, indicating these responses were directed by CD4⁺ T cells, while the response to STAM2^{P144L} was due to CD8⁺ T cell reactivity. Blockade by pan-MHC Class 11 and HLA-DR-specific antibodies confirmed that recognition of MVP^{L221F}, ARFGEF1^{P1337L}, and TENM1^{G443R} each occurred via CD4⁺ T cell activation in the context of HLA-DR loaded with neoantigen peptide (FIG. 17D). To further investigate whether CD4⁺ TIL were able to directly respond to the patient's tumor, we engineered the AT to enforce MHC Class II expression utilizing the Class II MHC Transactivator (CIITA) construct. CD4⁺ TIL responded to peptide-pulsed AT-CIITA via increased OX40 and 4-1BB expression on the cell surface as well as substantial effector molecule secretion, including the cytotoxic serine protease Granzyme B (FIG. 17E and FIG. 16D). Altogether these data indicated that CD4⁺ TIL from Patient 3 were able to recognize and respond to neoantigens presented directly on MHC Class II-competent tumor cells and APCs.

CD4⁺ TIL also dominated the neoantigen response observed in Patient 4. In addition to robust effector molecule secretion, the majority of CD4⁺ T cells upregulated OX40 and 4-1BB upon mutant peptide stimulation (FIGS. 5F and 5G). Following neoantigen-directed sorting, individual peptide screening indicated two neoantigen peptide hits (SLC15A4^{G71}S and NCLN^{P142H}) were responsible for the CD4⁺ TIL stimulation (FIG. 17H). The CD4⁺ T cell response was efficiently abrogated via HLA-DQ or HLA-DR blockade, indicative of the MHC Class II component restriction for SLC15A4^{G71s} and NCLN^{P142H}, respectively (FIG. 17I). CD4⁺ TIL were sorted for clonal enrichment in response to the individual neoantigen hits. Longitudinal clonal tracking of the two dominant sorted neoantigen-specific CD4⁺ TIL clones showed relative stability in vivo after ACT followed by a moderate dilution in clonal frequency over time (FIG. 17J). Analysis by scRNASeq showed substantial C1 (IL7R), C3 (activated) and C7 (Th2-like) cluster presence within the profiles of each of these clones. Only the SLC15A4^{G71S}-specific CD4⁺ clone clustered into C4 and C5, indicative of activated and cytotoxic T cells, although the relatively small clonal size of the NCLN^{P142H}-specific CD4⁺ TIL clone may have influenced this cluster distribution (FIG. 17K). The effector profile and strong persistence of the SLC15A4^{G71S}-specific CD4⁺ TIL indicated that this clone was therapeutically relevant following ACT, although overall this patient did not respond to the TIL infusion.

### c) Discussion

Detection of antigen-specific CD4⁺ T cells within the tumor microenvironment and circulating in the periphery of cancer patients led to early case studies investigating their efficacy in ACT. While these studies reported impressive initial successes harnessing this T cell population, CD4⁺ T cells remain an underexplored source of potent and capable anti-tumor effectors. Here, we studied the TIL infusion product from previously treated metastatic melanoma patients to better understand the nature of neoantigen-specific CD4⁺ TIL. Using a series of case studies, we investigated the CD4⁺ TIL compartment from four individuals previously treated with ACT utilizing TIL at MCC, each having a varying response to therapy and composition of infusion product. We discovered between one and six previously undetected neoantigen-specific CD4⁺ TIL clones per patient (1-4%), in line with other reports employing similar methodologies across multiple tumor types. Notably, 12 of the 13 neoantigen-specific clones detected were CD4⁺ T cells. Yossef and colleagues found a similar dominance of neoantigen reactivity by CD4⁺ T cells in metastatic epithelial cancer, indicating that CD4⁺ TIL are predisposed to detection and response to this particular type of tumor antigen. Tumor reactivity against AT and HLA-matched tumor was previously detected in the CD8-dominant TIL products of Patients 2-4, indicating that the CD8⁺ T cells present predominantly recognize shared antigens. Current standards within the field are ineffective and inefficient in terms of identification and inclusion of antigen-specific CD4⁺ T cells within traditional ACT products utilizing TIL, reinforcing the need to continue to develop strategies to investigate their presence and utility.

The identified neoantigen-specific CD4⁺ TIL responded to tumor antigens primarily via secretion of the effector molecules IFNg, TNFa, and Granzyme B. Additionally, these CD4⁺ TIL displayed direct anti-tumor cytotoxic capacity, which underscored their immense immunotherapeutic potential. We demonstrated that both MHC Class II-competent tumor cells and APCs were able to efficiently induce an effector response from neoantigen-specific CD4⁺ TIL, indicating that each interaction play a role in vivo during the T cell-mediated anti-tumor immune response. Endogenous or induced expression of MHC Class II proteins represent readily available targets for tumor-specific CD4⁺ TIL and consequently a mechanism for direct tumor cell lysis. Oh and colleagues reported similar findings regarding the ability of CD4⁺ T cells isolated from bladder cancer to kill tumor cells in an MHC Class II and Granzyme B-dependent manner, as implicated in our present study.

Neoantigens were prioritized on expression at the RNA level and theoretical binding affinity to each patient's respective HLA molecules for practicality. As such, additional tumor-specific CD4⁺ TIL clones may have been present within the TIL product and potentially excluded. We demonstrated an ability to select and expand these neoantigen-specific CD4⁺ TIL based on induction of OX40 and 4-1BB, accepted markers of tumor antigen recognition. Infusion of a TIL product enriched with neoantigen-specific CD4⁺ TIL represents a highly personalized therapy, especially as all immunogenic mutations discovered here and across other studies were found to be unique. Inclusion of neoantigen-specific CD4⁺ T cells into TIL products which already contain tumor-specific CD8⁺ T cells effectively broadens the application of this therapeutic approach. Improved selection of TIL based on antigen recognition ensures a highly specific and potent T cell product and can reduce the current standard of up to tens of billions of TIL for infusion. We also demonstrated that a neoantigen-reactive TCR isolated from CD4⁺ TIL can be utilized for generation of a highly potent TCR-T product. This represents an additional strategy for immunotherapeutic application of neoantigen-specific CD4⁺ T cells, with broader implications depending on the antigen.

Clonal tracking revealed a rapid increase in relative frequency for the majority of analyzed neoantigen-specific CD4⁺ TIL clones in the periphery of patients following ACT. These data support the hypothesis that tumor-reactive CD4⁺ T cells underwent clonal expansion upon recognition of cognate mutated peptides in vivo. This pattern was consistent between high and low frequency infused TIL clones, indicating a functional response rather than homeostatic proliferation. Specifically in Patient 1, the CD4⁺ TIL clone that recognized S100A11^{Q22R} peaked at nearly 40% of the entire peripheral blood T cell repertoire. Coupled with substantial neoantigen expression in the resected tumor, a robust clonal CD4⁺ T cell effector profile, and a lack of detectable CD8⁺ T cell reactivity, we surmised that this neoantigen-specific clone contributed to the regression of this patient's tumors and resultant CR. Similarly, the infusion product for Patients 2 and 4 contained neoantigen-specific CD4⁺ TIL clones that transiently increased in relative frequency, again indicating antigen detection and expansion in vivo. Overall, the maintenance of tumor-reactive CD4⁺ T cell clones in the periphery following TIL transfer demonstrated persistence of these clones during a critical interval for therapeutic response. Persistence of transferred T cells has been implicated in positive outcomes for patients receiving ACT of TIL and CAR T Cells, including the detection of activated cytotoxic CD4⁺ T cells in long-term responders.

Clonal analysis by scRNASeq supported the specificity and function of the discovered neoantigen-specific CD4⁺ TIL clones. The S100A11^{Q22R}-reactive CD4⁺ TIL from Patient 1 demonstrated a profile marked by relative enrichment of activated and cytotoxic T cells as well as elevated transcripts associated with neoantigen specificity. Similarly, neoantigen-specific CD4⁺ clones isolated from Patients 2 and 4 also clustered into activated effector T cell subsets, which corresponded with direct anti-tumor responses in vitro. In addition to cytolytic and cytokine gene modules, S100A11^{Q22R}-specific CD4⁺ TIL from Patient 1 expressed high levels of LAG-3, MHC Class II molecules, and chemokines. LAG-3 functions as a direct co-inhibitor for CD4⁺ T cells via impaired peptide-MHC Class II interactions and is therefore a marker of antigen experience and tumor-specific T cells. Targeting LAG-3 can serve as a strategy to release negative regulation and further enrich for neoantigen-reactive CD4⁺ T cells. MHC Class 11 and LAG-3 expression are also associated with T cell exhaustion possibly as a consequence of repeated antigen exposure, though relatively few neoantigen-specific CD4⁺ TIL clustered into the exhausted state by scRNASeq. Induction of MHC Class II on CD4⁺ T cells has been observed following T cell activation and indicates that CD4⁺ TIL is able to present MHC Class II peptides. The conditions and application of this mechanism require further interrogation but are supported by the GSEA data herein. Neoantigen-specific induction of chemokine production by CD4⁺ TIL indicated that these T cells can orchestrate a larger immune response, including attraction of CD8⁺ T cells, additional CD4⁺ T cells, DCs, and macrophages. Surprisingly, we did not detect substantial expression of CXCL13 at the RNA level amongst our neoantigen-reactive CD4⁺ TIL clones, as recently reported, indicating that tertiary lymphoid structure (TLS) formation is not critical to the anti-tumor activity here. Notably, the neoantigen-specific CD4⁺ TIL clones demonstrated a high degree of similarity between clusters, indicating a distinct profile of the neoantigen-specific CD4⁺ T cells when compared to other CD4⁺ T cell clones from the same patients.

We discovered undetected and uncharacterized clones of CD4⁺ TIL, which demonstrated evidence of tumor-reactivity in vitro and in vivo. Our retrospective analysis supports a functional role for neoantigen-specific CD4⁺ TIL in patient responses to ACT and underscores the importance of identifying these cells for enrichment and inclusion in TIL infusion products.

### E. References

Aibar S, González-Blas C B, Moerman T, Huynh-Thu V A, Imrichova H, Hulselmans G, Rambow F, Marine J-C, Geurts P, Aerts J, van den Oord J, Atak Z K, Wouters J, Aerts S. SCENIC: single-cell regulatory network inference and clustering. Nature Methods. 2017; 14(11):1083-6. doi: 10.1038/nmeth.4463.
Alspach E, Lussier D M, Miceli A P, Kizhvatov I, DuPage M, Luoma A M, Meng W, Lichti C F, Esaulova E, Vomund A N, Runci D, Ward J P, Gubin M M, Medrano RFV, Arthur C D, White J M, Sheehan KCF, Chen A, Wucherpfennig K W, Jacks T, Unanue E R, Artyomov M N, Schreiber R D. MHC-I I neoantigens shape tumour immunity and response to immunotherapy. Nature. 2019; 574(7780):696-701. Epub 20191023. doi: 10.1038/s41586-019-1671-8. PubMed PMID: 31645760; PMCID: PMC6858572.
Anadon C M, Yu X, Hanggi K, Biswas S, Chaurio R A, Martin A, Payne K K, Mandal G, Innamarato P, Harro C M, Mine J A, Sprenger K B, Cortina C, Powers J J, Costich T L, Perez B A, Gatenbee C D, Prabhakaran S, Marchion D, Heemskerk MHM, Curiel T J, Anderson A R, Wenham R M, Rodriguez P C, Conejo-Garcia J R. Ovarian cancer immunogenicity is governed by a narrow subset of progenitor tissue-resident memory T cells. Cancer Cell. 2022; 40(5):545-57 e13. Epub doi: 10.1016/j.ccell.2022.03.008. PubMed PMID: 35427494; PMCID: PMC9096229.
Anders S, Pyl P T, Huber W. HTSeq-a Python framework to work with high-throughput sequencing data. Bioinformatics. 2015; 31(2):166-9. Epub 20140925. doi: 10.1093/bioinformatics/btu638. PubMed PMID: 25260700; PMCID: PMC4287950.
Andreatta M, Corria-Osorio J, Miller S, Cubas R, Coukos G, Carmona S J. Interpretation of T cell states from single-cell transcriptomics data using reference atlases. Nature Communications. 2021; 12(1):2965. doi: 10.1038/s41467-021-23324-4.
Aydin A M, Hall M, Bunch B L, Branthoover H, Sannasardo Z, Mackay A, Beatty M, Samaik A A, Mullinax J E, Spiess P E, Pilon-Thomas S. Expansion of tumor-infiltrating lymphocytes (TIL) from penile cancer patients. Int Immunopharmacol. 2021; 94:107481. Epub 20210223. doi: 10.1016/j.intimp.2021.107481. PubMed PMID: 33636562; PMCID: PMC8205103.
Bai Y, Wang D, Fury W. PHLAT: Inference of High-Resolution HLA Types from RNA and Whole Exome Sequencing. Methods Mol Biol. 2018; 1802:193-201. doi: 10.1007/978-1-4939-8546-3_13. PubMed PMID: 29858810.
Bailey S R, Nelson M H, Majchrzak K, Bowers J S, Wyatt M M, Smith A S, Neal L R, Shirai K, Carpenito C, June C H, Zilliox M J, Paulos C M. Human CD26(high) T cells elicit tumor immunity against multiple malignancies via enhanced migration and persistence. Nat Commun. 2017; 8(1):1961. Epub 20171206. doi: 10.1038/s41467-017-01867-9. PubMed PMID: 29213079; PMCID: PMC5719008.
Besser M J, Shapira-Frommer R, Itzhaki O, Treves A J, Zippel D B, Levy D, Kubi A, Shoshani N, Zikich D, Ohay on Y, Ohayon D, Shalmon B, Markel G, Yerushalmi R, Apter S, Ben-Nun A, Ben-Ami E, Shimoni A, Nagler A, Schachter J. Adoptive transfer of tumor-infiltrating lymphocytes in patients with metastatic melanoma: intent-to-treat analysis and efficacy after failure to prior immunotherapies. Clin Cancer Res. 2013; 19(17):4792-800. Epub 20130520. doi: 10.1158/1078-CCR-13-0380. PubMed PMID: 23690483.
Cachot A, Bilous M, Liu Y C, Li X, Saillard M, Cenerenti M, Rockinger G A, Wyss T, Guillaume P, Schmidt J, Genolet R, Ercolano G, Protti M P, Reith W, Ioannidou K, de Leval L, Trapani J A, Coukos G, Harari A, Speiser D E, Mathis A, Gfeller D, Altug H, Romero P, Jandus C. Tumor-specific cytolytic CD4 T cells mediate immunity against human cancer. Sci Adv. 2021; 7(9). Epub doi: 10.1126/sciadv.abe3348. PubMed PMID: 33637530; PMCID: PMC7909889.
Cafri G, Yossef R, Pasetto A, Deniger D C, Lu Y C, Parkhurst M, Gartner J J, Jia L, Ray S, Ngo L T, Jafferji M, Sachs A, Prickett T, Robbins P F, Rosenberg S A. Memory T cells targeting oncogenic mutations detected in peripheral blood of epithelial cancer patients. Nat Commun. 2019; 10(1):449. Epub 20190125. doi: 10.1038/s41467-019-08304-z. PubMed PMID: 30683863; PMCID: PMC6347629.
Castellino F, Huang A Y, Altan-Bonnet G, Stoll S, Scheinecker C, Germain R N. Chemokines enhance immunity by guiding naive CD8⁺ T cells to sites of CD4⁺ T cell-dendritic cell interaction. Nature. 2006; 440(7086):890-5. doi: 10.1038/nature04651. PubMed PMID: 16612374.
Chacon J A, Samaik A A, Chen J Q, Creasy C, Kale C, Robinson J, Weber J, Hwu P, Pilon-Thomas S, Radvanyi L. Manipulating the tumor microenvironment ex vivo for enhanced expansion of tumor-infiltrating lymphocytes for adoptive cell therapy. Clin Cancer Res. 2015; 21(3):611-21. Epub 20141203. doi: 10.1158/1078-0432.CCR-14-1934. PubMed PMID: 25472998; PMCID: PMC4315752.
Cibulskis K, Lawrence M S, Carter S L, Sivachenko A, Jaffe D, Sougnez C, Gabriel S, Meyerson M, Lander E S, Getz G. Sensitive detection of somatic point mutations in impure and heterogeneous cancer samples. Nat Biotechnol. 2013; 31(3):213-9. Epub 20130210. doi: 10.1038/nbt.2514. PubMed PMID: 23396013; PMCID: PMC3833702.
DePristo M A, Banks E, Poplin R, Garimella K V, Maguire J R, Hartl C, Philippakis A A, del Angel G, Rivas M A, Hanna M, McKenna A, Fennell T J, Kernytsky A M, Sivachenko A Y, Cibulskis K, Gabriel S B, Altshuler D, Daly M J. A framework for variation discovery and genotyping using next-generation DNA sequencing data. Nat Genet. 2011; 43(5):491-8. Epub 20110410. doi: 10.1038/ng.806. PubMed PMID: 21478889; PMCID: PMC3083463.
Ding Z C, Huang L, Blazar B R, Yagita H, Mellor A L, Munn D H, Zhou G. Polyfunctional CD4(+) T cells are essential for eradicating advanced B-cell lymphoma after chemotherapy. Blood. 2012; 120(11):2229-39. Epub 20120802. doi: 10.1182/blood-2011-12-398321. PubMed PMID: 22859605; PMCID: PMC3447781.
Dobin A, Davis C A, Schlesinger F, Drenkow J, Zaleski C, Jha S, Batut P, Chaisson M, Gingeras T R. STAR: ultrafast universal RNA-seq aligner. Bioinformatics. 2013; 29(1):15-21. Epub doi: 10.1093/bioinformatics/bts635. PubMed PMID: 23104886; PMCID: PMC3530905.
Dudley M E, Gross C A, Somerville R P, Hong Y, Schaub N P, Rosati S F, White D E, Nathan D, Restifo N P, Steinberg S M, Wunderlich J R. Kammula U S, Sherry R M, Yang J C, Phan G Q, Hughes M S, Laurencot C M, Rosenberg S A. Randomized selection design trial evaluating CD8′-enriched versus unselected tumor-infiltrating lymphocytes for adoptive cell therapy for patients with melanoma. J Clin Oncol. 2013; 31(17):2152-9. Epub 20130506. doi: 10.1200/JCO.2012.46.6441. PubMed PMID: 23650429; PMCID: PMC3731980.
Fauskanger M, Haabeth OAW, Skjeldal F M, Bogen B, Tveita A A. Tumor Killing by CD4(+) T Cells Is Mediated via Induction of Inducible Nitric Oxide Synthase-Dependent Macrophage Cytotoxicity. Front Immunol. 2018; 9:1684. Epub 20180723. doi: 10.3389/fimmu.2018.01684. PubMed PMID: 30083157; PMCID: PMC6064871.
Germain P, Lun A, Macnair W, Robinson M. Doublet identification in single-cell sequencing data using scDblFinder [version 1; peer review: 1 approved, 1 approved with reservations]. F1000Research. 2021; 10(979). doi: 10.12688/fl000research.73600.1.
Godec J, Tan Y, Liberzon A, Tamayo P, Bhattacharya S, Butte A J, Mesirov J P, Haining W N. Compendium of Immune Signatures Identifies Conserved and Species-Specific Biology in Response to Inflammation. Immunity. 2016; 44(1):194-206. Epub 2016/01/12. doi: 10.1016/j.immuni.2015.12.006. PubMed PMID: 26795250.
Goding S R, Wilson K A, Xie Y, Harris K M, Baxi A, Akpinarli A, Fulton A, Tamada K, Strome S E, Antony P A. Restoring immune function of tumor-specific CD4⁺ T cells during recurrence of melanoma. J Immunol. 2013; 190(9):4899-909. Epub 20130327. doi: 10.4049/jimmunol.1300271. PubMed PMID: 23536636; PMCID: PMC3633733.
Haabeth OAW, Fauskanger M, Manzke M, Lundin K U, Corthay A, Bogen B, Tveita A A. CD4(+) T-cell-Mediated Rejection of MHC Class I I-Positive Tumor Cells Is Dependent on Antigen Secretion and Indirect Presentation on Host APCs. Cancer Res. 2018; 78(16):4573-85. Epub 20180511. doi: 10.1158/0008-5472.CAN-17-2426. PubMed PMID: 29752262.
Hall M, Liu H, Malafa M, Centeno B, Hodul P J, Pimiento J, Pilon-Thomas S, Sarnaik A A. Expansion of tumor-infiltrating lymphocytes (TIL) from human pancreatic tumors. J Immunother Cancer. 2016; 4:61. Epub 20161018. doi: 10.1186/s40425-016-0164-7. PubMed PMID: 27777771; PMCID: PMC5067894.
Hall M S, Mullinax J E, Cox C A, Hall A M, Beatty M S, Blauvelt J, Innamarato P, Nagle L, Branthoover H, Wiener D, Schachner B, Martinez A J, Richards A D, Rich C J, Colon Colon M, Schell M J, Teer J K, Khushalani N I, Weber J S, Mule J J, Sondak V K, Pilon-Thomas S, Samaik A A. Combination Nivolumab, CD137 Agonism, and Adoptive Cell Therapy with Tumor-Infiltrating Lymphocytes for Patients with Metastatic Melanoma. Clin Cancer Res. 2022. Epub doi: 10.1158/1078-0432.CCR-22-2103. PubMed PMID: 36215121.
Hanada K I, Zhao C, Gil-Hoyos R, Gartner J J, Chow-Parmer C, Lowery F J, Krishna S, Prickett T D, Kivitz S, Parkhurst M R, Wong N, Rae Z, Kelly M C, Goff S L, Robbins P F, Rosenberg S A, Yang J C. A phenotypic signature that identifies neoantigen-reactive T cells in fresh human lung cancers. Cancer Cell. 2022; 40(5):479-93 e6. Epub 20220421. doi: 10.1016/j.ccell.2022.03.012. PubMed PMID: 35452604.
Hao Y, Hao S, Andersen-Nissen E, Mauck W M, 3rd, Zheng S, Butler A, Lee M J, Wilk A J, Darby C, Zager M, Hoffman P, Stoeckius M, Papalexi E, Mimitou E P, Jain J, Srivastava A, Stuart T, Fleming L M, Yeung B, Rogers A J, McElrath J M, Blish C A, Gottardo R, Smibert P, Satija R. Integrated analysis of multimodal single-cell data. Cell. 2021; 184(13):3573-87 e29. Epub 2021/06/02. doi: 10.1016/j.cell.2021.04.048. PubMed PMID: 34062119; PMCID: PMC8238499.
Huang J, Khong H T, Dudley M E, El-Gamil M, Li Y F, Rosenberg S A, Robbins P F. Survival, persistence, and progressive differentiation of adoptively transferred tumor-reactive T cells associated with tumor regression. J Immunother. 2005; 28(3):258-67. doi: 10.1097/01.cji.0000158855.92792.7a. PubMed PMID: 15838383; PMCID: PMC2174599.
Hui-Yuen J, McAllister S, Koganti S, Hill E, Bhaduri-McIntosh S. Establishment of Epstein-Barr virus growth-transformed lymphoblastoid cell lines. J Vis Exp. 2011(57). Epub 20111108. doi: 10.3791/3321. PubMed PMID: 22090023; PMCID: PMC3308597.
Hunder N N, Wallen H, Cao J, Hendricks D W, Reilly J Z, Rodmyre R, Jungbluth A, Gnjatic S, Thompson J A, Yee C. Treatment of metastatic melanoma with autologous CD4⁺ T cells against N Y-ESO-1. N Engl J Med. 2008; 358(25):2698-703. doi: 10.1056/NEJMoa0800251. PubMed PMID: 18565862; PMCID: PMC3277288.
Innamarato P, Kodumudi K, Asby S, Schachner B, Hall M, Mackay A, Wiener D, Beatty M, Nagle L, Creelan B C, Sarnaik A A, Pilon-Thomas S. Reactive Myelopoiesis Triggered by Lymphodepleting Chemotherapy Limits the Efficacy of Adoptive T Cell Therapy. Mol Ther. 2020; 28(10):2252-70. Epub 20200624. doi: 10.1016/j.ymthe.2020.06.025. PubMed PMID: 32615068; PMCID: PMC7544980.
Janssen E M, Lemmens E E, Wolfe T, Christen U, von Herrath M G, Schoenberger S P. CD4⁺ T cells are required for secondary expansion and memory in CD8⁺ T lymphocytes. Nature. 2003; 421(6925):852-6. Epub 20030209. doi: 10.1038/nature01441. PubMed PMID: 12594515.
Jurtz V, Paul S, Andreatta M, Marcatili P, Peters B, Nielsen M. NetMHCpan-4.0: Improved Peptide-MHC Class I Interaction Predictions Integrating Eluted Ligand and Peptide Binding Affinity Data. JImmunol. 2017; 199(9):3360-8. Epub 20171004. doi: 10.4049/jimmunol.1700893. PubMed PMID: 28978689; PMCID: PMC5679736.
Khan O, Giles J R, McDonald S, Manne S, Ngiow S F, Patel K P, Werner M T, Huang A C, Alexander K A, Wu J E, Attanasio J, Yan P, George S M, Bengsch B, Staupe R P, Donahue G, Xu W, Amaravadi R K, Xu X, Karakousis G C, Mitchell T C, Schuchter L M, Kaye J, Berger S L, Wherry E J. TOX transcriptionally and epigenetically programs CD8⁺ T cell exhaustion. Nature. 2019; 571(7764):211-8. doi: 10.1038/s41586-019-1325-x.
Kohli K, Pillarisetty V G, Kim T S. Key chemokines direct migration of immune cells in solid tumors. Cancer Gene Ther. 2022; 29(1):10-21. Epub 20210218. doi: 10.1038/s41417-021-00303-x. PubMed PMID: 33603130; PMCID: PMC8761573.
Korotkevich G, Sukhov V, Budin N, Shpak B, Artyomov M N, Sergushichev A. Fast gene set enrichment analysis. bioRxiv. 2021:060012. doi: 10.1101/060012.
Li H, Durbin R. Fast and accurate short read alignment with Burrows-Wheeler transform. Bioinformatics. 2009; 25(14):1754-60. Epub 20090518. doi: 10.1093/bioinformatics/btp324. PubMed PMID: 19451168; PMCID: PMC2705234.
Liberzon A, Birger C, Thorvaldsdóttir H, Ghandi M, Mesirov J P, Tamayo P. The Molecular Signatures Database (MSigDB) hallmark gene set collection. Cell systems. 2015; 1(6):417-25. doi: 10.1016/j.cels.2015.12.004. PubMed PMID: 26771021.
Liberzon A, Subramanian A, Pinchback R, Thorvaldsdottir H, Tamayo P, Mesirov J P. Molecular signatures database (MSigDB) 3.0. Bioinformatics. 2011; 27(12):1739-40. doi: 10.1093/bioinformatics/btr260.
Linnemann C, van Buuren M M, Bies L, Verdegaal E M, Schotte R, Calis J J, Behjati S, Velds A, Hilkmann H, Atmioui D E, Visser M, Stratton M R, Haanen J B, Spits H, van der Burg S H, Schumacher T N. High-throughput epitope discovery reveals frequent recognition of neo-antigens by CD4⁺ T cells in human melanoma. Nat Med. 2015; 21(1):81-5. Epub 20141222. doi: 10.1038/nm.3773. PubMed PMID: 25531942.
Lowery F J, Krishna S, Yossef R, Parikh N B, Chatani P D, Zacharakis N, Parkhurst M R, Levin N, Sindiri S, Sachs A, Hitscherich K J, Yu Z, Vale N R, Lu Y C, Zheng Z, Jia L, Gartner J J, Hill V K, Copeland A R, Nah S K, Masi R V, Gasmi B, Kivitz S, Paria B C, Florentin M, Kim S P, Hanada K I, Li Y F, Ngo L T, Ray S, Shindorf M L, Levi S T, Shepherd R, Toy C, Parikh A Y, Prickett T D, Kelly M C, Beyer R, Goff S L, Yang J C, Robbins P F, Rosenberg S A. Molecular signatures of antitumor neoantigen-reactive T cells from metastatic human cancers. Science. 2022; 375(6583):877-84. Epub 20220203. doi: 10.1126/science.abl5447. PubMed PMID: 35113651.
Lu Y C, Jia L, Zheng Z, Tran E, Robbins P F, Rosenberg S A. Single-Cell Transcriptome Analysis Reveals Gene Signatures Associated with T-cell Persistence Following Adoptive Cell Therapy. Cancer Immunol Res. 2019; 7(11):1824-36. Epub 20190904. doi: 10.1158/2326-6066.CIR-19-0299. PubMed PMID: 31484655; PMCID: PMC6825592.
Lu Y C, Parker L L, Lu T, Zheng Z, Toomey M A, White D E, Yao X, Li Y F, Robbins P F, Feldman S A, van der Bruggen P, Klebanoff C A, Goff S L, Sherry R M, Kammula U S, Yang J C, Rosenberg S A. Treatment of Patients With Metastatic Cancer Using a Major Histocompatibility Complex Class I I-Restricted T-Cell Receptor Targeting the Cancer Germline Antigen MAGE-A3. J Clin Oncol. 2017; 35(29):3322-9. Epub 20170815. doi: 10.1200/JCO.2017.74.5463. PubMed PMID: 28809608; PMCID: PMC5652397.
Lun A T, McCarthy D J, Marioni J C. A step-by-step workflow for low-level analysis of single-cell RNA-seq data with Bioconductor. F1000Res. 2016; 5:2122. doi: 10.12688/fl000research.9501.2. PubMed PMID: 27909575; PMCID: PMC5112579.
Malekzadeh P, Yossef R, Cafri G, Paria B C, Lowery F J, Jafferji M, Good M L, Sachs A, Copeland A R, Kim S P, Kivitz S, Parkhurst M R, Robbins P F, Ray S, Xi L, Raffeld M, Yu Z, Restifo N P, Somerville RPT, Rosenberg S A, Deniger D C. Antigen Experienced T Cells from Peripheral Blood Recognize p53 Neoantigens. Clin Cancer Res. 2020; 26(6):1267-76. Epub 20200129. doi: 10.1158/1078-0432.CCR-19-1874. PubMed PMID: 31996390; PMCID: PMC7424598.
Mangalam A, Rodriguez M, David C. Role of MHC class I I expressing CD4⁺ T cells in proteolipid protein(91-110)-induced EAE in HLA-DR3 transgenic mice. Eur J Immunol. 2006; 36(12):3356-70. doi: 10.1002/eji.200636217. PubMed PMID: 17125142.
Maruhashi T, Okazaki I M, Sugiura D, Takahashi S, Maeda T K, Shimizu K, Okazaki T. LAG-3 inhibits the activation of CD4(+) T cells that recognize stable pMHCII through its conformation-dependent recognition of pMHCII. Nat Immunol. 2018; 19(12):1415-26. Epub 20181022. doi: 10.1038/s41590-018-0217-9. PubMed PMID: 30349037.
Matsuzaki J, Gnjatic S, Mhawech-Fauceglia P, Beck A, Miller A, Tsuji T, Eppolito C, Qian F, Lele S, Shrikant P, Old L J, Odunsi K. Tumor-infiltrating N Y-ESO-1-specific CD8⁺ T cells are negatively regulated by LAG-3 and PD-1 in human ovarian cancer. Proc Natl Acad Sci USA. 2010; 107(17):7875-80. Epub 20100412. doi: 10.1073/pnas.1003345107. PubMed PMID: 20385810; PMCID: PMC2867907.
Mautner J, Jaffee E M, Pardoll D M. Tumor-specific CD4⁺ T cells from a patient with renal cell carcinoma recognize diverse shared antigens. Int J Cancer. 2005; 115(5):752-9. doi: 10.1002/ijc.20927. PubMed PMID: 15704175.
McGinnis C S, Murrow L M, Gartner Z J. DoubletFinder: Doublet Detection in Single-Cell RNA Sequencing Data Using Artificial Nearest Neighbors. Cell Syst. 2019; 8(4):329-37 e4. Epub 2019/04/08. doi: 10.1016/j.cels.2019.03.003. PubMed PMID: 30954475; PMCID: PMC6853612.
Melenhorst J J, Chen G M, Wang M, Porter D L, Chen C, Collins M A, Gao P, Bandyopadhyay S, Sun H, Zhao Z, Lundh S, Pruteanu-Malinici I, Nobles C L, Maji S, Frey N V, Gill S I, Tian L, Kulikovskaya I, Gupta M, Ambrose D E, Davis M M, Fraietta J A, Brogdon J L, Young R M, Chew A, Levine B L, Siegel D L, Alanio C, Wherry E J, Bushman F D, Lacey S F, Tan K, June C H. Decade-long leukaemia remissions with persistence of CD4(+) CAR T cells. Nature. 2022; 602(7897):503-9. Epub 20220202. doi: 10.1038/s41586-021-04390-6. PubMed PMID: 35110735.
Mucida D, Husain M M, Muroi S, van Wijk F, Shinnakasu R, Naoe Y, Reis B S, Huang Y, Lambolez F, Docherty M, Attinger A, Shui J W, Kim G, Lena C J, Sakaguchi S, Miyamoto C, Wang P, Atarashi K, Park Y, Nakayama T, Honda K, Ellmeier W, Kronenberg M, Taniuchi I, Cheroutre H. Transcriptional reprogramming of mature CD4(+) helper T cells generates distinct MHC class I I-restricted cytotoxic T lymphocytes. Nat Immunol. 2013; 14(3):281-9. Epub doi: 10.1038/ni.2523. PubMed PMID: 23334788; PMCID: PMC3581083.
Mullinax J E, Hall M, Prabhakaran S, Weber J, Khushalani N, Eroglu Z, Brohl A S, Markowitz J, Royster E, Richards A, Stark V, Zager J S, Kelley L, Cox C, Sondak V K, Mule J J, Pilon-Thomas S, Sarnaik A A. Combination of Ipilimumab and Adoptive Cell Therapy with Tumor-Infiltrating Lymphocytes for Patients with Metastatic Melanoma. Front Oncol. 2018; 8:44. Epub 20180302. doi: 10.3389/fonc.2018.00044. PubMed PMID: 29552542; PMCID: PMC5840208.
Oh D Y, Kwek S S, Raju S S, Li T, McCarthy E, Chow E, Aran D, Ilano A, Pai C S, Rancan C, Allaire K, Burra A, Sun Y, Spitzer M H, Mangul S, Porten S, Meng M V, Friedlander T W, Ye C J, Fong L. Intratumoral CD4(+) T Cells Mediate Anti-tumor Cytotoxicity in Human Bladder Cancer. Cell. 2020; 181(7):1612-25 e13. Epub 20200603. doi: 10.1016/j.cell.2020.05.017. PubMed PMID: 32497499; PMCID: PMC7321885.
Oja A E, Piet B, van der Zwan D, Blaauwgeers H, Mensink M, de Kivit S. Borst J, Nolte M A, van Lier RAW, Stark R, Hombrink P. Functional Heterogeneity of CD4(+) Tumor-Infiltrating Lymphocytes With a Resident Memory Phenotype in NSCLC. Front Immunol. 2018; 9:2654. Epub 20181116. doi: 10.3389/fimmu.2018.02654. PubMed PMID: 30505306; PMCID: PMC6250821.
Parkhurst M, Gros A, Pasetto A, Prickett T, Crystal J S, Robbins P, Rosenberg S A. Isolation of T-Cell Receptors Specifically Reactive with Mutated Tumor-Associated Antigens from Tumor-Infiltrating Lymphocytes Based on CD137 Expression. Clin Cancer Res. 2017; 23(10):2491-505. Epub 20161108. doi: 10.1158/1078-0432.CCR-16-2680. PubMed PMID: 27827318; PMCID: PMC6453117.
Parkhurst M R, Robbins P F, Tran E, Prickett T D, Gartner J J, Jia L, Ivey G, Li Y F, El-Gamil M, Lalani A, Crystal J S, Sachs A, Groh E, Ray S, Ngo L T, Kivitz S, Pasetto A, Yossef R, Lowery F J, Goff S L, Lo W, Cafri G, Deniger D C, Malekzadeh P, Ahmadzadeh M, Wunderlich J R, Somerville RPT, Rosenberg S A. Unique Neoantigens Arise from Somatic Mutations in Patients with Gastrointestinal Cancers. Cancer Discov. 2019; 9(8):1022-35. Epub 20190604. doi: 10.1158/2159-8290.CD-18-1494. PubMed PMID: 31164343; PMCID: PMC7138461.
Pearce H, Hutton P, Chaudhri S, Porfiri E, Patel P, Viney R. Moss P. Spontaneous CD4(+) and CD8(+) T-cell responses directed against cancer testis antigens are present in the peripheral blood of testicular cancer patients. Eur J Immunol. 2017; 47(7):1232-42. Epub 20170626. doi: 10.1002/eji.201646898. PubMed PMID: 28555838; PMCID: PMC5519936.
Pilon-Thomas S, Kuhn L, Ellwanger S, Janssen W, Royster E, Marzban S, Kudchadkar R, Zager J, Gibney G, Sondak V K, Weber J, Mule J J, Samaik A A. Efficacy of adoptive cell transfer of tumor-infiltrating lymphocytes after lymphopenia induction for metastatic melanoma. J Immunother. 2012; 35(8):615-20. doi: 10.1097/CJI.0b013e31826e8f5f. PubMed PMID: 22996367; PMCID: PMC4467830.
Prieto P A, Durflinger K H, Wunderlich J R, Rosenberg S A, Dudley M E. Enrichment of CD8⁺ cells from melanoma tumor-infiltrating lymphocyte cultures reveals tumor reactivity for use in adoptive cell therapy. J Immunother. 2010; 33(5):547-56. doi: 10.1097/CJI.0b013e3181d367bd. PubMed PMID: 20463593; PMCID: PMC6309789.
Quezada S A, Simpson T R, Peggs K S, Merghoub T, Vider J, Fan X, Blasberg R, Yagita H, Muranski P, Antony P A, Restifo N P, Allison J P. Tumor-reactive CD4(+) T cells develop cytotoxic activity and eradicate large established melanoma after transfer into lymphopenic hosts. J Exp Med. 2010; 207(3):637-50. Epub 20100215. doi: 10.1084/jem.20091918. PubMed PMID: 20156971; PMCID: PMC2839156.
Radvanyi L G, Bernatchez C, Zhang M, Fox P S, Miller P, Chacon J, Wu R, Lizee G, Mahoney S, Alvarado G, Glass M, Johnson V E, McMannis J D, Shpall E, Prieto V, Papadopoulos N, Kim K, Homsi J, Bedikian A, Hwu W J, Patel S, Ross M I, Lee J E, Gershenwald J E, Lucci A, Royal R, Cormier J N, Davies M A, Mansaray R, Fulbright O J, Toth C, Ramachandran R, Wardell S, Gonzalez A, Hwu P. Specific lymphocyte subsets predict response to adoptive cell therapy using expanded autologous tumor-infiltrating lymphocytes in metastatic melanoma patients. Clin Cancer Res. 2012; 18(24):6758-70. Epub 20121002. doi: 10.1158/1078-0432.CCR-12-1177. PubMed PMID: 23032743; PMCID: PMC3525747.
Ramello M C, Benzaid I, Kuenzi B M, Lienlaf-Moreno M, Kandell W M, Santiago D N, Pabon-Saldana M, Darville L, Fang B, Rix U, Yoder S, Berglund A, Koomen J M, Haura E B, Abate-Daga D. An immunoproteomic approach to characterize the CAR interactome and signalosome. Sci Signal. 2019; 12(568). Epub 20190212. doi: 10.1126/scisignal.aap9777. PubMed PMID: 30755478; PMCID: PMC6506216.
Reynisson B, Alvarez B, Paul S, Peters B, Nielsen M. NetMHCpan-4.1 and NetMHCIIpan-4.0: improved predictions of MHC antigen presentation by concurrent motif deconvolution and integration of M S MHC eluted ligand data. Nucleic Acids Res. 2020; 48(W1):W449-W54. doi: 10.1093/nar/gkaa379. PubMed PMID: 32406916; PMCID: PMC7319546.
Rosemblit C, Datta J, Lowenfeld L, Xu S, Basu A, Kodumudi K, Wiener D, Czemiecki B J. Oncodriver inhibition and CD4(+) Th1 cytokines cooperate through Statl activation to induce tumor senescence and apoptosis in HER2+ and triple negative breast cancer: implications for combining immune and targeted therapies. Oncotarget. 2018; 9(33):23058-77. Epub 20180501. doi: 10.18632/oncotarget.25208. PubMed PMID: 29796172; PMCID: PMC5955413.
Rosenberg S A, Yang J C, Sherry R M, Kammula U S, Hughes M S, Phan G Q, Citrin D E, Restifo N P, Robbins P F, Wunderlich J R, Morton K E, Laurencot C M, Steinberg S M, White D E, Dudley M E. Durable complete responses in heavily pretreated patients with metastatic melanoma using T-cell transfer immunotherapy. Clin Cancer Res. 2011; 17(13):4550-7. Epub 20110415. doi: 10.1158/1078-0432.CCR-11-0116. PubMed PMID: 21498393; PMCID: PMC3131487.
Samaik A, Hall M, Mullinax J, Royster E, Richards A, Crago G, Zager J, Vernon S, Weber J, Pilon-Thomas S. Clinical results of combined vemurafenib and tumor-infiltrating lymphocyte therapy for metastatic melanoma. Journal for ImmunoTherapy of Cancer. 2015; 3(S2). doi: 10.1186/2051-1426-3-s2-p49.
Satija R, Farrell J A, Gennert D, Schier A F, Regev A. Spatial reconstruction of single-cell gene expression data. Nat Biotechnol. 2015; 33(5):495-502. doi: 10.1038/nbt.3192. PubMed PMID: 25867923; PMCID: PMC4430369.
Saunders C T, Wong W S, Swamy S, Becq J, Murray L J, Cheetham R K. Strelka: accurate somatic small-variant calling from sequenced tumor-normal sample pairs. Bioinformatics. 2012; 28(14):1811-7. Epub 20120510. doi: 10.1093/bioinformatics/bts271. PubMed PMID:
Shedlock D J, Shen H. Requirement for CD4 T cell help in generating functional CD8 T cell memory. Science. 2003; 300(5617):337-9. doi: 10.1126/science.1082305. PubMed PMID: 12690201.
Subramanian A, Tamayo P, Mootha V K, Mukherjee S, Ebert B L, Gillette M A, Paulovich A, Pomeroy S L, Golub T R, Lander E S, Mesirov J P. Gene set enrichment analysis: A knowledge-based approach for interpreting genome-wide expression profiles. Proceedings of the National Academy of Sciences. 2005; 102(43):15545-50. doi: 10.1073/pnas.0506580102.
Szabo P A, Levitin H M, Miron M, Snyder M E, Senda T, Yuan J, Cheng Y L, Bush E C, Dogra P, Thapa P, Farber D L, Sims P A. Single-cell transcriptomics of human T cells reveals tissue and activation signatures in health and disease. Nature Communications. 2019; 10(1):4706. doi: 10.1038/s41467-019-12464-3.
Szolek A, Schubert B, Mohr C, Sturm M, Feldhahn M, Kohlbacher O. OptiType: precision HLA typing from next-generation sequencing data. Bioinformatics. 2014; 30(23):3310-6. Epub doi: 10.1093/bioinformatics/btu548. PubMed PMID: 25143287; PMCID: PMC4441069.
Tawbi H A, Schadendorf D, Lipson E J, Ascierto P A, Matamala L, Castillo Gutierrez E, Rutkowski P, Gogas H J, Lao C D, De Menezes J J, Dalle S, Arance A, Grob J J, Srivastava S, Abaskharoun M, Hamilton M, Keidel S, Simonsen K L, Sobiesk A M, Li B, Hodi F S, Long G V, Investigators R-. Relatlimab and Nivolumab versus Nivolumab in Untreated Advanced Melanoma. N Engl J Med. 2022; 386(1):24-34. doi: 10.1056/NEJMoa2109970. PubMed PMID: 34986285.
Tempero R M, VanLith M L, Morikane K, Rowse G J, Gendler S J, Hollingsworth M A. CD4⁺ Lymphocytes Provide MUC1-Specific Tumor Immunity In Vivo That Is Undetectable In Vitro and Is Absent in MUC1 Transgenic Mice. The Journal of Immunology. 1998; 161(10):5500-6.
Tran E, Turcotte S, Gros A, Robbins P F, Lu Y C, Dudley M E, Wunderlich J R, Somerville R P, Hogan K, Hinrichs C S, Parkhurst M R, Yang J C, Rosenberg S A. Cancer immunotherapy based on mutation-specific CD4⁺ T cells in a patient with epithelial cancer. Science. 2014; 344(6184):641-doi: 10.1126/science.1251102. PubMed PMID: 24812403; PMCID: PMC6686185.
Veatch J R, Lee S M, Shasha C, Singhi N, Szeto J L, Moshiri A S, Kim T S, Smythe K, Kong P, Fitzgibbon M, Jesernig B, Bhatia S, Tykodi S S, Hall E T, Byrd D R, Thompson J A, Pillarisetty V G, Duhen T, McGarry Houghton A, Newell E, Gottardo R, Riddell S R. Neoantigen-specific CD4(+) T cells in human melanoma have diverse differentiation states and correlate with CD8(+) T cell, macrophage, and B cell function. Cancer Cell. 2022; 40(4):393-409 e9. doi: 10.1016/j.ccell.2022.03.006. PubMed PMID: 35413271; PMCID: PMC9011147.
Wang K, Li M. Hakonarson H. ANNOVAR: functional annotation of genetic variants from high-throughput sequencing data. Nucleic Acids Res. 2010; 38(16):e164. Epub 20100703. doi: 10.1093/nar/gkg603. PubMed PMID: 20601685; PMCID: PMC2938201.
Wolock S L, Lopez R, Klein A M. Scrublet: Computational Identification of Cell Doublets in Single-Cell Transcriptomic Data. Cell Syst. 2019; 8(4):281-91 e9. Epub 2019/04/08. doi: 10.1016/j.cels.2018.11.005. PubMed PMID: 30954476; PMCID: PMC6625319.
Xie Y, Akpinarli A, Maris C, Hipkiss E L, Lane M, Kwon E K, Muranski P, Restifo N P. Antony P A. Naive tumor-specific CD4(+) T cells differentiated in vivo eradicate established melanoma. J Exp Med. 2010; 207(3):651-67. Epub 20100215. doi: 10.1084/jem.20091921. PubMed PMID: 20156973; PMCID: PMC2839147.
Yossef R, Tran E, Deniger D C, Gros A, Pasetto A, Parkhurst M R, Gartner J J, Prickett T D, Cafri G, Robbins P F, Rosenberg S A. Enhanced detection of neoantigen-reactive T cells targeting unique and shared oncogenes for personalized cancer immunotherapy. JCI Insight. 2018; 3(19). Epub 20181004. doi: 10.1172/jci.insight.122467. PubMed PMID: 30282837; PMCID: PMC6237474.
Zacharakis N, Chinnasamy H, Black M, Xu H, Lu Y C, Zheng Z, Pasetto A, Langhan M, Shelton T, Prickett T, Gartner J, Jia L, Trebska-McGowan K, Somerville R P, Robbins P F, Rosenberg S A, Goff S L, Feldman S A. Immune recognition of somatic mutations leading to complete durable regression in metastatic breast cancer. Nat Med. 2018; 24(6):724-30. Epub 20180604. doi: 10.1038/s41591-018-0040-8. PubMed PMID: 29867227; PMCID: PMC6348479.
Zheng C, Fass J N, Shih Y P, Gunderson A J, Sanjuan Silva N, Huang H, Bernard B M, Rajamanickam V, Slagel J, Bifulco C B, Piening B, Newell PHA, Hansen P D, Tran E. Transcriptomic profiles of neoantigen-reactive T cells in human gastrointestinal cancers. Cancer Cell. 2022; 40(4):410-23 e7. doi: 10.1016/j.ccell.2022.03.005. PubMed PMID: 35413272.
Zheng L, Qin S, Si W, Wang A, Xing B, Gao R, Ren X, Wang L, Wu X. Zhang J, Wu N, Zhang N, Zheng H, Ouyang H, Chen K, Bu Z, Hu X, Ji J, Zhang Z. Pan-cancer single-cell landscape of tumor-infiltrating T cells. Science. 2021; 374(6574):abe6474. doi: doi:10.1126/science.abe6474.

1. An engineered CD4+ T cell comprising a T cell receptor (TCR) alpha (TCRα) chain comprising a complimentary determining region (CDR) 3 (CDR3) as set forth in SEQ ID NO: 1, SEQ ID NO: 11, SEQ ID NO: 17, SEQ ID NO: 23, SEQ ID NO:29, SEQ ID NO: 35, or SEQ ID NO: 41.

2. The engineered CD4+ T cell of claim 1, wherein the TCRα CDR3 is encoded by the nucleic acid as set forth in SEQ ID NO: 2, SEQ ID NO: 12, SEQ ID NO: 18, SEQ ID NO: 24, SEQ ID NO: 30, SEQ ID NO: 36, or SEQ ID NO: 42.

3. The engineered CD4+ T cell of claim 1, wherein the TCRα is encoded by the nucleic acid as set forth in SEQ ID NO: 5, SEQ ID NO: 15, SEQ ID NO: 21, SEQ ID NO: 27, SEQ ID NO: 33, SEQ ID NO: 39, or SEQ ID NO: 45.

4. The engineered CD4+ T cell of claim 1, further comprising a TCR beta (TCRβ) chain comprising a CDR3 as set forth in SEQ ID NO: 3, SEQ ID NO: 13, SEQ ID NO: 19, SEQ ID NO: 25, SEQ ID NO: 31, SEQ ID NO: 37, or SEQ ID NO: 43.

5. The engineered CD4+ T cell of claim 4, wherein the TCRβ CDR3 is encoded by the nucleic acid as set forth in SEQ ID NO: 4, SEQ ID NO: 14, SEQ ID NO: 20, SEQ ID NO: 26, SEQ ID NO: 32, SEQ ID NO: 38, or SEQ ID NO: 44.

6. The engineered CD4+ T cell of claim 4, wherein the TCRβ is encoded by the nucleic acid as set forth in SEQ ID NO: 6, SEQ ID NO: 16, SEQ ID NO: 22, SEQ ID NO: 28, SEQ ID NO: 34, SEQ ID NO: 40, or SEQ ID NO: 46.

7. An engineered CD4+ T cell comprising a T cell receptor beta (TCRβ) chain comprising a complimentary determining region 3 (CDR3) as set forth in SEQ ID NO: 3.

8. The engineered CD4+ T cell of claim 7, wherein the TCRβ CDR3 is encoded by the nucleic acid as set forth in SEQ ID NO: 4, SEQ ID NO: 14, SEQ ID NO: 20, SEQ ID NO: 26, SEQ ID NO: 32, SEQ ID NO: 38, or SEQ ID NO: 44.

9. The engineered CD4+ T cell of claim 7, wherein the TCRβ is encoded by the nucleic acid as set forth in SEQ ID NO: 6, SEQ ID NO: 16, SEQ ID NO: 22, SEQ ID NO: 28, SEQ ID NO: 34, SEQ ID NO: 40, or SEQ ID NO: 46.

10. A method of treating a cancer in a subject comprising administering to the subject one or more of the engineered CD4+ T cells of claim 1.

11. A method of treating a cancer in a subject comprising administering to the subject an adoptive cell therapy comprising one or more engineered CD4+ T cells.

12. The method of treating a cancer of claim 11, wherein the one or more engineered CD4+ T cells comprise a T cell receptor alpha (TCRα) chain comprising a complimentary determining region (CDR) 3 (CDR3) as set forth in SEQ ID NO: 1, SEQ ID NO: 11, SEQ ID NO: 17, SEQ ID NO: 23, SEQ ID NO:29, SEQ ID NO: 35, or SEQ ID NO: 41.

13. The method of treating a cancer of claim 12, wherein the TCRα CDR3 of the engineered CD4+ T cell is encoded by the nucleic acid as set forth in SEQ ID NO: 2, SEQ ID NO: 12, SEQ ID NO: 18, SEQ ID NO: 24, SEQ ID NO: 30, SEQ ID NO: 36, or SEQ ID NO: 42.

14. The method of treating a cancer of claim 12, wherein the TCRα of the engineered CD4+ T cell is encoded by the nucleic acid as set forth in SEQ ID NO: 5, SEQ ID NO: 15, SEQ ID NO: 21, SEQ ID NO: 27, SEQ ID NO: 33, SEQ ID NO: 39, or SEQ ID NO: 45.

15. The method of treating a cancer of claim 12, further comprising a TCR beta (TCRβ) chain comprising a CDR3 as set forth in SEQ ID NO: 3, SEQ ID NO: 13, SEQ ID NO: 19, SEQ ID NO: 25, SEQ ID NO: 31, SEQ ID NO: 37, or SEQ ID NO: 43.

16. The method of treating a cancer of claim 15, wherein the TCRβ CDR3 of the engineered CD4+ T cell is encoded by the nucleic acid as set forth in SEQ ID NO: 4, SEQ ID NO: 14, SEQ ID NO: 20, SEQ ID NO: 26, SEQ ID NO: 32, SEQ ID NO: 38, or SEQ ID NO: 44.

17. The method of treating a cancer of claim 15, wherein the TCRβ is encoded by the nucleic acid as set forth in SEQ ID NO: 6, SEQ ID NO: 16, SEQ ID NO: 22, SEQ ID NO: 28, SEQ ID NO: 34, SEQ ID NO: 40, or SEQ ID NO: 46.

18. The method of treating a cancer of claim 11, wherein the one or more engineered CD4+ T cells comprise a T cell receptor beta (TCRβ) chain comprising a complimentary determining region 3 (CDR3) as set forth in SEQ ID NO: 3, SEQ ID NO: 13, SEQ ID NO: 19, SEQ ID NO: 25, SEQ ID NO: 31, SEQ ID NO: 37, or SEQ ID NO: 43.

19. The method of treating a cancer of claim 18, wherein the TCRβ CDR3 of the engineered CD4+ T cell is encoded by the nucleic acid as set forth in SEQ ID NO: 4, SEQ ID NO: 14, SEQ ID NO: 20, SEQ ID NO: 26, SEQ ID NO: 32, SEQ ID NO: 38, or SEQ ID NO: 44.

20. The method of treating a cancer of claim 18, wherein the TCRβ of the engineered CD4+ T cell is encoded by the nucleic acid as set forth in SEQ ID NO: 6, SEQ ID NO: 16, SEQ ID NO: 22, SEQ ID NO: 28, SEQ ID NO: 34, SEQ ID NO: 40, or SEQ ID NO: 46.

21. The method of treating a cancer of claim 11, wherein the CD4+ T cells are obtained from tumor infiltrating lymphocytes (TILs), marrow infiltrating lymphocytes (MILs), memory CD4+ T cells, Th1 CD4+ T cells, Th17 CD4+ T cells, Th2 CD4+ T cells, and regulatory CD4+ T cells (Tregs).

22. The method of treating a cancer of claim 11, wherein at least 80% of the cells transferred are CD4+ T cells.

23. The method of treating a cancer of claim 11, wherein the engineered CD4+ T cells are cultured in the presence of autologous tumor prior to administration to the subject.


SEQUENCE LISTING (46 sequences)

SEQ ID NO: 1 - protein, 14 aa, synthetic construct
CALDFTGGGNKLTF

SEQ ID NO: 2 - DNA, 42 bp, synthetic construct
  source 1..42: mol_type = other DNA
tgtgctctagatttcacgggaggaggaaacaaactcaccttt

SEQ ID NO: 3 - protein, 16 aa, synthetic construct
CASSPSTGGIGDTQYF

SEQ ID NO: 4 - DNA, 48 bp, synthetic construct
  source 1..48: mol_type = other DNA
tgtgccagctcaccctcgacaggggggattggagatacgcagtatttt

SEQ ID NO: 5 - DNA, 678 bp, synthetic construct
  source 1..678: mol_type = other DNA
gagtatttcctaccccctcagagcctgtctcagatgtgaaacgaagttcactgcatagctggattaggccagtatgtgtaaggggctgaacaggcttgccattgattggctggataggaaggccagaacttccttctaggggtagaagaaccccagtaacacctatcaaactaaacagaatggctttttggctgagaaggctgggtctacattttaggccacatttggggagacgaatggagtcattcctgggaggtgttttgctgattttgtggcttcaagtggactgggtgaagagccaaaagatagaacagaattccgaggccctgaacattcaggagggtaaaacggccaccctgacctgcaactatacaaactattctccagcatacttacagtggtaccgacaagatccaggaagaggccctgttttcttgctacttatacgtgaaaatgagaaagaaaaaaggaaagaaagactgaaggtcacctttgataccacccttaaacagagtttgtttcatatcacagcctcccagcctgcagactcagctacctacctctgtgctctagatttcacgggaggaggaaacaaactcacctttgggacaggcactcagctaaaagtggaactcaatatccagaaccctgaccctgccgtgtaccagctgagagact

SEQ ID NO: 6 - DNA, 539 bp, synthetic construct
  source 1..539: mol_type = other DNA
agctgtgaggtctggttccccgacgtgctgcagcaagtgcctttgccctgcctgtgggctccctccatggccaactctgctatggacaccagagtactctgctgtgcggtcatctgtcttctgggggcaggtctctcaaatgccggcgtcatgcagaacccaagacacctggtcaggaggaggggacaggaggcaagactgagatgcagcccaatgaaaggacacagtcatgtttactggtatcggcagctcccagaggaaggtctgaaattcatggtttatctccagaaagaaaatatcatagatgagtcaggaatgccaaaggaacgattttctgctgaatttcccaaagagggccccagcatcctgaggatccagcaggtagtgcgaggagattcggcagcttatttctgtgccagctcaccctcgacaggggggattggagatacgcagtattttggcccaggcacccggctgacagtgctcgaggacctgaaaaacgtgttcccacccgaggtcgctgtgtttgagccatcaga

SEQ ID NO: 7 - DNA, 405 bp, synthetic construct
  source 1..405: mol_type = other DNA
atggacaccagagtgctgtgctgcgccgtgatctgcctgctgggcgccggcctgagcaacgccggcgtgatgcagaaccccagacacctggtgaggaggaggggccaggaggccagactgagatgcagccccatgaagggccacagccacgtgtactggtacaggcagctgcccgaggagggcctgaagttcatggtgtacctgcagaaggagaacatcatcgacgagagcggcatgcccaaggagaggttcagcgccgagttccccaaggagggccccagcatcctgagaatccagcaggtagtgagaggagacagcgcagcatacttctgcgccagcagccccagcaccggcggcatcggcgacacccagtacttcggccccggcaccaggctgaccgtgctg

SEQ ID NO: 8 - DNA, 456 bp, synthetic construct
  source 1..456: mol_type = other DNA
atggccttctggctgagaaggctgggcctgcacttcaggccccacctgggcagaaggatggagagcttcctgggcggcgtgctgctgatcctgtggctgcaggtggactgggtgaagagccagaagatcgagcagaacagcgaggccctgaacatccaggagggcaagaccgccaccctgacctgcaactacaccaactacagccccgcctacctgcagtggtacaggcaggaccccggcagaggccccgtgttcctgctgctgatcagggagaacgagaaggagaagaggaaggagagactgaaggtgaccttcgacaccaccctgaagcagagcctgttccacatcaccgccagccagcccgccgacagcgccacctacctgtgcgccctggacttcaccggcggcggcaacaagctgaccttcggcaccggcacccagctgaaggtggagctg

SEQ ID NO: 9 - DNA, 1875 bp, synthetic construct
  source 1..1875: mol_type = other DNA
atggacaccagagtgctgtgctgcgccgtgatctgcctgctgggcgccggcctgagcaacgccggcgtgatgcagaaccccagacacctggtgaggaggaggggccaggaggccagactgagatgcagccccatgaagggccacagccacgtgtactggtacaggcagctgcccgaggagggcctgaagttcatggtgtacctgcagaaggagaacatcatcgacgagagcggcatgcccaaggagaggttcagcgccgagttccccaaggagggccccagcatcctgagaatccagcaggtagtgagaggagacagcgcagcatacttctgcgccagcagccccagcaccggcggcatcggcgacacccagtacttcggccccggcaccaggctgaccgtgctggaggatctgagaaacgtgaccccccctaaggtgagcctgtttgagccttccaaggccgagatcgccaataagcagaaggccacactggtgtgtctggcccgcggcttctttccagatcacgtggagctgtcttggtgggtgaacggcaaggaggtgcacagcggagtgtgcaccgacccacaggcctacaaggagtctaattacagctattgtctgagctccaggctgcgcgtgtccgccacattctggcacaaccccaggaatcacttccgctgccaggtgcagtttcacggcctgtctgaggaggataagtggcctgagggaagcccaaagccagtgacccagaacatctccgcagaggcatggggaagggcagactgtggcatcacctccgcctcttatcagcagggcgtgctgagcgccacaatcctgtacgagatcctgctgggcaaggccaccctgtatgccgtgctggtgagcacactggtggtcatggctatggtgaagagaaagaactccagggcaaagagaagcggatccggagccaccaatttctctctgctgaagcaggcaggcgacgtggaggagaaccctggaccaatggccttctggctgagaaggctgggcctgcacttcaggccccacctgggcagaaggatggagagcttcctgggcggcgtgctgctgatcctgtggctgcaggtggactgggtgaagagccagaagatcgagcagaacagcgaggccctgaacatccaggagggcaagaccgccaccctgacctgcaactacaccaactacagccccgcctacctgcagtggtacaggcaggaccccggcagaggccccgtgttcctgctgctgatcagggagaacgagaaggagaagaggaaggagagactgaaggtgaccttcgacaccaccctgaagcagagcctgttccacatcaccgccagccagcccgccgacagcgccacctacctgtgcgccctggacttcaccggcggcggcaacaagctgaccttcggcaccggcacccagctgaaggtggagctgaacatccagaaccccgagcccgccgtgtaccagctgaaggaccccagaagccaggacagcaccctgtgcctgttcaccgacttcgacagccagatcaacgtgcccaagacaatggagagcggcaccttcatcaccgacaagtgcgtgctggacatgaaggcaatggacagcaagagcaacggcgccatcgcctggagcaaccagaccagcttcacctgccaggacatcttcaaggagaccaacgccacctaccccagcagcgacgtgccctgcgacgccaccctgaccgagaagagcttcgagaccgacatgaacctgaacttccagaacctgctggtgatcgtgctgagaatcctgctgctgaaggtggccggcttcaacctgctgatgaccctgagactgtggagcagctga

SEQ ID NO: 10 - protein, 624 aa, synthetic construct
MDTRVLCCAVICLLGAGLSNAGVMQNPRHLVRRRGQEARLRCSPMKGHSHVYWYRQLPEEGLKFMVYLQKENIIDESGMPKERFSAEFPKEGPSILRIQQVVRGDSAAYFCASSPSTGGIGDTQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMAFWLRRLGLHFRPHLGRRMESFLGGVLLILWLQVDWVKSQKIEQNSEALNIQEGKTATLTCNYTNYSPAYLQWYRQDPGRGPVFLLLIRENEKEKRKERLKVTFDTTLKQSLFHITASQPADSATYLCALDFTGGGNKLTFGTGTQLKVELNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 11 - protein, 14 aa, synthetic construct
CAMREGDSNYQLIW

SEQ ID NO: 12 - DNA, 42 bp, synthetic construct
  source 1..42: mol_type = other DNA
tgtgcaatgagagagggcgatagcaactatcagttaatctgg

SEQ ID NO: 13 - protein, 15 aa, synthetic construct
CASRWDIQTTDTQYF

SEQ ID NO: 14 - DNA, 45 bp, synthetic construct
  source 1..45: mol_type = other DNA
tgtgccagcagatgggacatccaaaccacagatacgcagtatttt

SEQ ID NO: 15 - DNA, 523 bp, synthetic construct
  source 1..523: mol_type = other DNA
gccaggttcacttcacagtacagagtcctgaaaataaagaagaaaatttttttttatctagaaaaagaaccaaacatgtcactttctagcctgctgaaggtggtcacagcttcactgtggctaggacctggcattgcccagaagataactcaaacccaaccaggaatgttcgtgcaggaaaaggaggctgtgactctggactgcacatatgacaccagtgatccaagttatggtctattctggtacaagcagcccagcagtggggaaatgatttttcttatttatcaggggtcttatgaccagcaaaatgcaacagaaggtcgctactcattgaatttccagaaggcaagaaaatccgccaaccttgtcatctccgcttcacaactgggggactcagcaatgtatttctgtgcaatgagagagggcgatagcaactatcagttaatctggggcgctgggaccaagctaattataaagccagatatccagaaccctgaccctgccgtgtaccagctgagagact

SEQ ID NO: 16 - DNA, 495 bp, synthetic construct
  source 1..495: mol_type = other DNA
acctggagcccccagaactggcagacacctgcctgatgctgccatgggcccccagctccttggctatgtggtcctttgccttctaggagcaggccccctggaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagatgggacatccaaaccacagatacgcagtattttggcccaggcacccggctgacagtgctcgaggacctgaaaaacgtgttcccacccgaggtcgctgtgtttgagccatcaga

SEQ ID NO: 17 - protein, 17 aa, synthetic construct
CALSFPNYGGSQGNLIF

SEQ ID NO: 18 - DNA, 51 bp, synthetic construct
  source 1..51: mol_type = other DNA
tgtgctctgagtttcccgaattatggaggaagccaaggaaatctcatcttt

SEQ ID NO: 19 - protein, 17 aa, synthetic construct
CASSREDRGPALRTIYF

SEQ ID NO: 20 - DNA, 51 bp, synthetic construct
  source 1..51: mol_type = other DNA
tgtgccagcagtagagaggacaggggaccggctctccgcaccatatatttt

SEQ ID NO: 21 - DNA, 504 bp, synthetic construct
  source 1..504: mol_type = other DNA
gactgtgatttcttcatgttaaggatcaagaccattatttgggtaacacactaaagatgaactattctccaggcttagtatctctgatactcttactgcttggaagaacccgtggaaattcagtgacccagatggaagggccagtgactctctcagaagaggccttcctgactataaactgcacgtacacagccacaggatacccttcccttttctggtatgtccaatatcctggagaaggtctacagctcctcctgaaagccacgaaggctgatgacaagggaagcaacaaaggttttgaagccacataccgtaaagaaaccacttctttccacttggagaaaggctcagttcaagtgtcagactcagcggtgtacttctgtgctctgagtttcccgaattatggaggaagccaaggaaatctcatctttggaaaaggcactaaactctctgttaaaccaaatatccagaaccctgaccctgccgtgtaccagctgagagact

SEQ ID NO: 22 - DNA, 255 bp, synthetic construct
  source 1..255: mol_type = other DNA
attctttcttcaaagcagccatgggaatcaggctcctctgtcgtgtggccttttgtttcctggctgtaggcctcgtagatgtgaaagtaacccagagctcgagatatctagtcaaaaggacgggagagaaagtttttctggaatgtgtccaggatatggaccatgaaaatatgttctggtatcgacaagacccaggtctggggctacggctgatctatttctcatatgatgttaaaatgaaagaaaaaggagata

SEQ ID NO: 23 - protein, 14 aa, synthetic construct
CILRAQTGANNLFF

SEQ ID NO: 24 - DNA, 42 bp, synthetic construct
  source 1..42: mol_type = other DNA
tgcatcctgagagctcaaactggggcaaacaacctcttcttt

SEQ ID NO: 25 - protein, 15 aa, synthetic construct
CASSLTGATNTEAFF

SEQ ID NO: 26 - DNA, 45 bp, synthetic construct
  source 1..45: mol_type = other DNA
tgtgccagcagccttacaggggccactaacactgaagctttcttt

SEQ ID NO: 27 - DNA, 650 bp, synthetic construct
  source 1..650: mol_type = other DNA
ggggggttcattctttttgccaccttgtgctttgtttatattttttccttacgaggagccttttctatctttggctgacgatttctggggagggggaaaattgaaacctgcctgatgtgggatgtgctgtggctgctgctttgttgcttgggacctcctctgacctaggatcagacacagagtctgagttctggggcctggaacctcaatgtgcacttgaacaatgaagttggtgacaagcattactgtactcctatctttgggtattatgggtgatgctaagaccacacagccaaattcaatggagagtaacgaagaagagcctgttcacttgccttgtaaccactccacaatcagtggaactgattacatacattggtatcgacagcttccctcccagggtccagagtacgtgattcatggtcttacaagcaatgtgaacaacagaatggcctctctggcaatcgctgaagacagaaagtccagtaccttgatcctgcaccgtgctaccttgagagatgctgctgtgtactactgcatcctgagagctcaaactggggcaaacaacctcttctttgggactggaacgagactcaccgttattccctatatccagaaccctgaccctgccgtgtaccagctgagagact

SEQ ID NO: 28 - DNA, 608 bp, synthetic construct
  source 1..608: mol_type = other DNA
gggttaccacttatttttatttggtggttgtaaaattttttccctggggggactgtgggaactgctctgtggcgacaaggacgtccctcatcctctgtttatggggacagtgaccctgatctggtaaagctcccatcctgccctgaccctgccatgggcaccagcctcctctgctggatggccctgtgtctcctgggggcagatcacgcagatactggagtctcccaggaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagccttacaggggccactaacactgaagctttctttggacaaggcaccagactcacagttgtagaggacctgaacaaggtgttcccacccgaggtcgctgtgtttgagccatcaga

SEQ ID NO: 29 - protein, 14 aa, synthetic construct
CAVSDPRYGQNFVF

SEQ ID NO: 30 - DNA, 42 bp, synthetic construct
  source 1..42: mol_type = other DNA
tgtgctgtgagtgatcctcgctatggtcagaattttgtcttt

SEQ ID NO: 31 - protein, 15 aa, synthetic construct
CASSLSRGKGYGYTF

SEQ ID NO: 32 - DNA, 45 bp, synthetic construct
  source 1..45: mol_type = other DNA
tgtgccagcagtctttctaggggaaagggctatggctacaccttc

SEQ ID NO: 33 - DNA, 570 bp, synthetic construct
  source 1..570: mol_type = other DNA
ttttgaaacccttcaaaggcagagacttgtccagcctaacctgcctgctgctcctagctcctgaggctcagggcccttggcttctgtccgctctgctcagggccctccagcgtggccactgctcagccatgctcctgctgctcgtcccagtgctcgaggtgatttttaccctgggaggaaccagagcccagtcggtgacccagcttggcagccacgtctctgtctctgagggagccctggttctgctgaggtgcaactactcatcgtctgttccaccatatctcttctggtatgtgcaataccccaaccaaggactccagcttctcctgaagtacacaacaggggccaccctggttaaaggcatcaacggttttgaggctgaatttaagaagagtgaaacctccttccacctgacgaaaccctcagcccatatgagcgacgcggctgagtacttctgtgctgtgagtgatcctcgctatggtcagaattttgtctttggtcccggaaccagattgtccgtgctgccctatatccagaaccctgaccctgccgtgtaccagctgagagact

SEQ ID NO: 34 - DNA, 518 bp, synthetic construct
  source 1..518: mol_type = other DNA
gagagtcctgctcccctttcatcaatgcacagatacagaagacccctccgtcatgcagcatctgccatgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagtctttctaggggaaagggctatggctacaccttcggttcggggaccaggttaaccgttgtagaggacctgaacaaggtgttcccacccgaggtcgctgtgtttgagccatcaga

SEQ ID NO: 35 - protein, 15 aa, synthetic construct
CAALNYGGSQGNLIF

SEQ ID NO: 36 - DNA, 45 bp, synthetic construct
  source 1..45: mol_type = other DNA
tgtgctgccttgaattatggaggaagccaaggaaatctcatcttt

SEQ ID NO: 37 - protein, 15 aa, synthetic construct
CASSQDARGANVLTF

SEQ ID NO: 38 - DNA, 45 bp, synthetic construct
  source 1..45: mol_type = other DNA
tgcgccagcagccaagacgcgcgaggggccaacgtcctgactttc

SEQ ID NO: 39 - DNA, 670 bp, synthetic construct
  source 1..670: mol_type = other DNA
gctcttccgatctggctcgaaggacattacataaaccgttgtggtgtatggggaagtaaaactgtaaatgttcttaagtgtgcatttctgctgcttctgatgggctgaaaatcccctttgatttctaaagtaaatgtagagacgttttaaaaataaaggactcctttgtccaagatatattccgaaatcctccaacagagacctgtgtgagcttctgctgcagtaataatggtgaagatccggcaatttttgttggctattttgtggcttcagctaagctgtgtaagtgccgccaaaaatgaagtggagcagagtcctcagaacctgactgcccaggaaggagaatttatcacaatcaactgcagttactcggtaggaataagtgccttacactggctgcaacagcatccaggaggaggcattgtttccttgtttatgctgagctcagggaagaagaagcatggaagattaattgccacaataaacatacaggaaaagcacagctccctgcacatcacagcctcccatcccagagactctgccgtctacatctgtgctgccttgaattatggaggaagccaaggaaatctcatctttggaaaaggcactaaactctctgttaaaccaaatatccagaaccctgaccctgccgtgtaccagctgagagact

SEQ ID NO: 40 - DNA, 481 bp, synthetic construct
  source 1..481: mol_type = other DNA
agaggccccatctcagacccgaggctagcatgggctgcaggctgctctgctgtgcggttctctgtctcctgggagcagttcccatagacactgaagttacccagacaccaaaacacctggtcatgggaatgacaaataagaagtctttgaaatgtgaacaacatatggggcacagggctatgtattggtacaagcagaaagctaagaagccaccggagctcatgtttgtctacagctatgagaaactctctataaatgaaagtgtgccaagtcgcttctcacctgaatgccccaacagctctctcttaaaccttcacctacacgccctgcagccagaagactcagccctgtatctctgcgccagcagccaagacgcgcgaggggccaacgtcctgactttcggggccggcagcaggctgaccgtgctggaggacctgaaaaacgtgttcccacccgaggtcgctgtgtttgagccatcaga

SEQ ID NO: 41 - protein, 14 aa, synthetic construct
CVAALNTGGFKTIF

SEQ ID NO: 42 - DNA, 42 bp, synthetic construct
  source 1..42: mol_type = other DNA
tgtgtggcggcgcttaatactggaggcttcaaaactatcttt

SEQ ID NO: 43 - protein, 15 aa, synthetic construct
CASSEIGLAVGEQFF

SEQ ID NO: 44 - DNA, 45 bp, synthetic construct
  source 1..45: mol_type = other DNA
tgtgccagcagtgagattggactagcggtgggtgagcagttcttc

SEQ ID NO: 45 - DNA, 481 bp, synthetic construct
  source 1..481: mol_type = other DNA
agtcaacttctgggagcagatctctgcagaataaaaatgaaaaagcatctgacgaccttcttggtgattttgtggctttatttttatagggggaatggcaaaaaccaagtggagcagagtcctcagtccctgatcatcctggagggaaagaactgcactcttcaatgcaattatacagtgagccccttcagcaacttaaggtggtataagcaagatactgggagaggtcctgtttccctgacaatcatgactttcagtgagaacacaaagtcgaacggaagatatacagcaactctggatgcagacacaaagcaaagctctctgcacatcacagcctcccagctcagcgattcagcctcctacatctgtgtggcggcgcttaatactggaggcttcaaaactatctttggagcaggaacaagactatttgttaaagcaaatatccagaaccctgaccctgccgtgtaccagctgagagact

SEQ ID NO: 46 - DNA, 509 bp, synthetic construct
  source 1..509: mol_type = other DNA
gttcccctatcaccgatgcacagacccagaagacccctccatcctgtagcacctgccatgagcatcgggctcctgtgctgtgtggccttttctctcctgtgggcaagtccagtgaatgctggtgtcactcagaccccaaaattccaggtcctgaagacaggacagagcatgacactgcagtgtgcccaggatatgaaccataactccatgtactggtatcgacaagacccaggcatgggactgaggctgatttattactcagcttctgagggtaccactgacaaaggagaagtccccaatggctacaatgtctccagattaaacaaacgggagttctcgctcaggctggagtcggctgctccctcccagacatctgtgtacttctgtgccagcagtgagattggactagcggtgggtgagcagttcttcgggccagggacacggctcaccgtgctagaggacctgaaaaacgtgttcccacccgaggtcgctgtgtttgagccatcaga